(19) United States
(12) Patent Application Publication
(10) Pub. No.: US 2022/0289791 A1
(43) Pub. Date: Sep. 15, 2022

(54) LACTATE DEHYDROGENASE INHIBITOR POLYPEPTIDES FOR USE IN THE TREATMENT OF CANCER

(71) Applicant: UNIVERSITÉ CATHOLIQUE DE LOUVAIN, Louvain-la-Neuve (BE)

(72) Inventors: Pierre SONVEAUX, Sterrebeek (BE); Raphaël FRÉDÉRICK, Godinne (BE); Léopold THABAULT, Ixelles (BE); Lucie BRISSON, Tours (FR); Tamara COPETTI, Trieste (IT)

(73) Assignee: UNIVERSITÉ CATHOLIQUE DE LOUVAIN, Louvain-la-Neuve (BE)

(21) Appl. No.: 17/608,002

(22) PCT Filed: Apr. 30, 2020
(86) PCT No.: PCT/EP2020/062141

§ 371 Date: Nov. 1, 2021

(30) Foreign Application Priority Data
May 2, 2019 (EP) 19172347.7
Mar. 6, 2020 (EP) 20161569.7

(51) Int. Cl. C07K 7/06; C12N 9/04; A61P 35/00

(52) U.S. Cl. CPC C07K 7/06; C12N 9/0006; A61P 35/00; C12Y 101/01027; A61K 38/00

(57) ABSTRACT

A polypeptide that modulates the activity of at least one isoform of the native tetrameric lactate dehydrogenase, and the use thereof as a medicament for the treatment of a cancer. More particularly, linear and cyclic polypeptides that inhibit the tetramerization of the lactate dehydrogenase subunits, and compositions and kits including the polypeptides.

### FIELD OF INVENTION

The present invention relates to a polypeptide that modulates the activity of native tetrameric lactate dehydrogenase, and the use thereof as a medicament for the treatment of a cancer. More particularly, the invention relates to linear and cyclic polypeptides that inhibit the tetramerization of the lactate dehydrogenase subunits.

### BACKGROUND OF INVENTION

Cancer cells undergo tremendous metabolic adaptation in order to sustain their anabolic growth and proliferative agenda. The most distinctive feature of this metabolic plasticity is the amplified glycolytic activity and lactate production, regardless of oxygen availability. Known as the Warburg effect, this enhancement of glycolysis allows cancer cells to redirect a fraction of the carbohydrate flux from energy production to anabolic pathways, thereby strengthening cellular proliferation. On the other hand, the elevation of intra and extra-cellular lactate, the end-product of glycolysis, drives pathogenesis by promoting several phenomena such as angiogenesis (de Saedeleer et al. (2012); Beckert et al. (2006); Végran et al. (2011)), invasiveness (Izumi et al. (2011); Colen et al. (2011)) and inflammation (Colegio et al. (2014); Doherty and Cleveland (2013)). At the core of lactate metabolism, Lactate Dehydrogenase (LDH, EC:1.1.1.27), a NAD⁺-dependent enzyme, catalyzes the interconversion of pyruvate to lactate. Besides being directly implicated in the pathogenic pathways sub-mentioned, LDH allows for a metabolic symbiosis between oxidative and glycolytic cancer cells (Sonveaux et al. (2008)), promotes autophagy through lysosomal acidification (Brisson et al. (2016)) as well as stabilizes the intracellular redox balance by regenerating NAD⁺. In addition, LDHA appears to be regulated by acetylation in cancer tissue. CN102805861 (FUDAN UNIVERSITY) provides activators of LDHA acetylation on amino acid residue K5. The recent discoveries of the wide implication of LDH in cancer pathogenesis thus makes it an appealing target for cancer therapy.

LDH is a tetrameric enzyme constituted of two main subunits, namely LDHA (also referred as to LDH-M subunit) and LDHB (also referred as to LDH-H subunit), which can assemble in functional homo or hetero tetramers resulting in 5 isoforms, namely LDH1, LDH2, LDH3, LDH4 and LDH5. Among these 5 isoforms, the homo-tetrameric LDH1 (4 LDHB subunits) and LDH5 (4 LDHA subunits) are the most extensively studied and are well known for their implications in cancer cell proliferation and survival through the mechanisms mentioned above.

Despite sharing a high structural identity, LDH1 and LDH5 differ in their localization as well as in their catalytic properties. LDH5 is predominantly found in glycolytic tissues such as skeletal muscles while LDH1 subunit is mainly expressed in heart, neurons and red blood cells. LDH5 also presents a higher affinity for pyruvate and a higher maximum velocity (Vmax) for pyruvate reduction compared to LDHB subunit (Eszes et al. (1996); Hewitt et al. (1999)). On the contrary, LDH1 subunit shows a better propensity in physiological and pathological conditions to oxidize lactate to pyruvate, allowing oxidative cells to use lactate as a nutrient source for oxidative phosphorylation and as an intracellular signaling agent.

Due to the broadly pathogenic implication of LDH in cancer cell proliferation and survival, intense efforts were devoted during the past years to develop small molecules able to selectively inhibit LDH activity (Rani and Kumar (2016)). For example, Döbeli et al. (1982) isolated two peptides from human urine that interfere with the assembly of catalytically inactive monomers to active tetrameric enzyme units, and Jafary et al. (2019) employed in silico methods to design inhibitory peptides for lactate dehydrogenase through the disturbance in tetramerization of the enzyme. Despite the different catalytic properties between LDH1 and LDH5, the catalytic site of the two tetrameric enzymes shares a high structural homology. As a result, achieving a high selectivity over one isoform to the other was found to be a challenging task with mitigated results (Labadie et al. (2015); Billiard et al. (2013); Rai et al. (2017)). Moreover, whether achieving a selectivity between isoforms is desirable or not is still under debate (Ždralević et al. (2018)). In fact, while some groups focus on developing selective inhibitors, others argue for the potential additional therapeutic value of a non-selective pan-LDH inhibitor (Purkey et al. (2016); Ward et al. (2012)). So far, all the molecules developed to inhibit LDH focused on an interaction at the catalytic site and therefore suffered from common drawbacks due to inherent structural features of LDH active site.

Indeed, the LDH catalytic site is highly polar and is mainly constituted by the co-factor binding site (Fiume et al. (2014)). As a result, most molecules interacting with LDH's active site are NAD+-competitive and interact therefore with LDH's “Rossman fold” (Ward et al. (2012); Kohlmann et al. (2013)). The “Rossman fold” is a structural motif shared by many dinucleotide-binding enzymes (Rao and Rossmann (1973)).

Consequently, most LDH inhibitors generally face a lack of selectivity towards other NAD⁺-dependent enzymes (Fiume et al. (2014)). On the other hand, molecules that achieve potent interaction with LDH1 or LDH5 catalytic site are usually hampered by their highly polar nature and, hence, non “drug-like” features often resulting in poor clinical value (Ward et al. (2012); Kohlmann et al. (2013)). Altogether, although LDH remains a very promising and validated target, LDH inhibitors have yet to demonstrate their potential in clinical trials.

LDH-inhibitors are the subject matter of the present invention.

### SUMMARY

This invention thus relates to a polypeptide that inhibits the tetramerization of the lactate dehydrogenase subunits, said polypeptide comprising an amino acid sequence of formula (I)

X1-X2-X3-X4-X5-X6-X7-X8 (I) (SEQ ID NO: 5),

wherein:
X1 represents any amino acid residue, preferentially selected from the group consisting of amino acid residues A, G, K and C;
X2 represents C, T or S;
X3 represents C, L, A, T, cpA (cyclopropyl-L-alanine), chG (L-cyclohexylglycine), chA (cyclohexyl-L-alanine) or mlL (γ-methyl-L-leucine);
X4 represents any amino acid residue, preferentially a positively charged or neutral amino acid residue, preferentially selected from the group consisting of amino acid residues K, C, A and Aib (2-aminoisobutyric acid), and more preferentially amino acid K;
X5 represents any amino acid residue, preferentially a negatively or positively charged or neutral amino acid residue, preferentially selected from the group consisting of amino acid residues E, D, K, A and C, and more preferentially amino acid E;
X6 represents any amino acid residue, preferentially a negatively or positively charged or neutral amino acid residue, preferentially selected from the group consisting of amino acid residues E, K, Q, A, Aib (2-aminoisobutyric acid) and C, and more preferentially amino acid K;
X7 represents C, L, I, cpA (cyclopropyl-L-alanine), chG (L-cyclohexylglycine), chA (cyclohexyl-L-alanine) or mlL (γ-methyl-L-leucine);
X8 represents C, I or G.

In some embodiments, the polypeptide of the invention is a linear polypeptide, preferentially comprising an amino acid sequence selected from the group consisting of SEQ ID NO: 6 to SEQ ID NO: 22. In some other embodiments, the polypeptide of the invention is a cyclic polypeptide, preferentially comprising an amino acid sequence selected from the group consisting of SEQ ID NO: 30 to SEQ ID NO: 35, SEQ ID NO: 55 to SEQ ID NO: 58, SEQ ID NO: 61 to SEQ ID NO: 65, SEQ ID NO: 67 and SEQ ID NO: 68. In certain embodiments, said cyclic polypeptide comprises an amino acid sequence selected from the group consisting of SEQ ID NO: 55, SEQ ID NO: 61, SEQ ID NO: 62, SEQ ID NO: 63, SEQ ID NO: 67 and SEQ ID NO: 68. In some embodiments, said cyclic polypeptide comprises an amino acid sequence represented by SEQ ID NO: 61, SEQ ID NO: 67 or SEQ ID NO: 68. In some embodiments, said lactate dehydrogenase subunit is lactate dehydrogenase B (LDHB) subunit. In some embodiment, the —OH group of the free —COOH group of the last amino acid residue at the C-terminus of the polypeptide is replaced by a group selected from an —O-alkyl group, an —O-aryl group, a —NH₂ group, a —N-alkyl amine group, a —N-aryl amine group or a —N-alkyl/aryl group.

The present invention further concerns a polynucleotide encoding a polypeptide according to the invention.

The present invention further concerns a pharmaceutical composition comprising at least one polypeptide according to the invention, and at least one pharmaceutically acceptable vehicle.

The present invention further concerns a kit for preventing and/or treating a cancer comprising at least one polypeptide, a polynucleotide or a pharmaceutical composition according to the invention, and optionally at least one anticancer agent.

The present invention further concerns a polypeptide, a polynucleotide, or a pharmaceutical composition for use as a medicament.

The present invention further concerns a polypeptide, a polynucleotide, or a pharmaceutical composition according to the invention for preventing and/or treating a cancer.

The present invention also relates to a method for screening a compound affecting the tetramerization of the lactate dehydrogenase subunits comprising the steps of:
a. providing a system comprising truncated lactate dehydrogenase (LDHtr) subunits;
b. providing the system with a candidate compound modulating the activity of a native tetrameric LDH;
c. measuring a level of binding of the candidate compound to a dimer of LDHtr subunits in the presence or in the absence of a polypeptide according to the invention;
   wherein the observation of a competition between the polypeptide and the candidate compound for the binding to the dimer of LDHtr subunits is indicative of the candidate compound being an inhibitor of the tetramerization of the lactate dehydrogenase subunits.

In one embodiment, the observation of a competition between the polypeptide and the candidate compound for the binding to the dimer of LDHBtr subunits is indicative of the specificity of the binding of the candidate compound towards the tetramerization site onto the lactate dehydrogenase subunits.

A further aspect of the invention pertains to a method for screening a compound affecting the tetramerization of the lactate dehydrogenase subunits comprising the steps of:
a. providing a system (1) comprising truncated lactate dehydrogenase (LDHtr) subunits and a system (2) comprising native tetrameric LDH;
b. providing the systems (1) and (2) with a candidate compound modulating the activity of a native tetrameric LDH;
c. measuring a level of binding (Kd) of the candidate compound to a dimer of LDHtr subunits in system (1) and to a native tetrameric LDH in system (2);
   wherein the observation of a binding of the candidate compound to the dimer of LDHtr subunits in system (1) and wherein the observation of an altered binding of the candidate compound to the native tetrameric LDH in system (2) are indicative of the candidate compound being an inhibitor of the tetramerization of the lactate dehydrogenase subunits, by interacting at the surface of the LDH subunits.

### Definitions

In the present invention, unless defined otherwise, the following terms have the following meanings:
The term “About”, when preceding a figure, means plus or less 10% of the value of said figure.
The term “amino acid substitution” refers to the replacement in a polypeptide of one amino acid with another amino acid. In one embodiment, an amino acid is replaced with another amino acid having similar structural and/or chemical properties, e.g. conservative amino acid replacements. “Conservative amino acid substitution” may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; negatively charged (acidic) amino acids include aspartic acid and glutamic acid. Non-conservative substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic) Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.
The term “polynucleotide” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Polynucleotide” refers to triple-stranded regions comprising RNA or DNA or both RNA and DNA. The term Polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “Polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.
The term “polypeptide” refers to refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.
The expression “preventing (a) cancer” is intended to mean keeping from happening at least one adverse effect or symptom of a cancer.
The term “subject” refers to a mammal, preferably a human. In one embodiment, the subject is a man. In another embodiment, the subject is a woman. In one embodiment, a subject may be a “patient”, i.e. a warm-blooded animal, more preferably a human, who/which is awaiting the receipt of, or is receiving medical care or was/is/will be the object of a medical procedure, or is monitored for the development of inflammation. In one embodiment, the subject is an adult (for example a subject above the age of 18). In another embodiment, the subject is a child (for example a subject below the age of 18).
The term “therapeutically effective amount” means the level or amount of agent that is aimed at, without causing significant negative or adverse side effects to the target, (1) delaying or preventing the onset of cancer; (2) slowing down or stopping the progression, aggravation, or deterioration of one or more symptoms of cancer; (3) bringing about ameliorations of the symptoms of cancer; (4) reducing the severity or incidence of cancer; or (5) preventing cancer formation. In one embodiment, a therapeutically effective amount is administered prior to the onset of cancer formation, for a prophylactic or preventive action.
The term “treating (a) cancer” or “treatment” or “alleviation” refers to both therapeutic treatment and prophylactic or preventative measures; wherein the object is to prevent or slow down (lessen) cancer. Those in need of treatment include those already with cancer as well as those prone to have cancer or those in whom cancer is to be prevented. A subject or mammal is successfully “treated” for a cancer if, after receiving a therapeutic amount of a polypeptide according to the present invention, the patient shows observable and/or measurable reduction in or absence of one or more of the following: reduction in the number of pathogenic cells; reduction in the percent of total cells that are pathogenic; and/or relief to some extent, one or more of the symptoms associated with cancer; reduced morbidity and mortality, and improvement in quality of life issues. The above parameters for assessing successful treatment and improvement in the disease are readily measurable by routine procedures familiar to a physician.

### DETAILED DESCRIPTION

With the aim to set out a novel approach to LDH inhibition, focus was brought on unravelling LDH allosteric sites whose targeting might result in unprecedented ways to approach this problem. Given that the tetramer is the minimal functional unit, LDH activity relies on both its catalytic site and oligomerization state. Regarding LDH, subunits are held together thanks to their N-terminal arms that extend from one subunit and wraps around two adjacent subunits, thus promoting the overall tetrameric cohesion. Interestingly, LDH 32 N-terminal amino-acids fragment is known to interfere in vitro with LDH tetramerization process (Döbeli et al. (1987)). Altogether, these observations prompted the inventors to evaluate this N-terminal arm as a starting point for the design and development of molecules interfering with LDH tetramerization.

This invention relates to a polypeptide that modulates the activity of at least one isoform of the native tetrameric lactate dehydrogenase.

By “lactate dehydrogenase” or “LDH”, it is meant a tetrameric enzyme that is capable of catalyzing the interconversion of pyruvate and lactate with concomitant interconversion of NADH and NAD⁺.

To date, 5 isoforms of lactate dehydrogenase, i.e. LDH1, LDH2, LDH3, LDH4 and LDH5, have been identified, which account for a peculiar combination of 2 subunits, namely the LDHA subunit and the LDHB subunit.

Within the context of the invention, by “modulating”, it is meant that the polypeptide of the invention has a biological effect of significantly up-regulating or down-regulating the biological activity of any one of the 5 isoforms of the lactate dehydrogenase, i.e. LDH1, LDH2, LDH5, LDH4 and LDH5 and or the biological activity of one or more subunit(s), i.e. the LDHA subunit and/or the LDHB subunit.

By “native”, it is meant that the sequence of lactate dehydrogenase (LDH), as referred to in the present application, is derived from nature, e.g., from any species. Further, such native sequence of lactate dehydrogenase can be isolated from nature or can be produced by recombinant or synthetic means from subunit LDHA and/or LDHB.

In some embodiments, the LDHA subunit is represented by an amino acid sequence SEQ ID NO: 1, and the LDHB subunit is represented by an amino acid sequence SEQ ID NO: 2.

In a particular embodiment, the polypeptide of the invention inhibits the activity of at least one isoform of the native tetrameric lactate dehydrogenase or at least one subunit thereof.

By “inhibitor” or “inhibiting”, it is meant that the polypeptide of the invention has for biological effect to inhibit or significantly reduce or down-regulate the biological activity of any one of the 5 isoforms of lactate dehydrogenase. In a particular embodiment, the polypeptide according to the invention is capable of inhibiting up to about 10%, preferably up to about 25%, preferably up to about 50%, preferably up to about 75%, 80%, 90%, 95%, more preferably up to about 96%, 97%, 98%, 99% or 100% of the activity of the native lactate dehydrogenase.

In an embodiment, the polypeptide of the invention inhibits the tetramerization of the lactate dehydrogenase subunits.

In some embodiment, the polypeptide of the invention inhibits the tetramerization of at least one of the 4 LDHA subunits, so as to inhibit the activity of isoform LDH5.

In some embodiment, the polypeptide of the invention inhibits the tetramerization of at least one of the 3 LDHA subunits and/or the LDHB subunit, so as to inhibit the activity of isoform LDH4.

In some embodiment, the polypeptide of the invention inhibits the tetramerization of at least one of the 2 LDHA subunits and/or at least one of the 2 LDHB subunits, so as to inhibit the activity of isoform LDH3.

In some embodiment, the polypeptide of the invention inhibits the tetramerization of the LDHA subunit and/or at least one of the 3 LDHB subunits so as to inhibit the activity of isoform LDH2.

In some embodiment, the polypeptide of the invention inhibits the tetramerization of at least one of the 4 LDHB subunits, so as to inhibit the activity of isoform LDH1.

It is needless to mention that the inhibition of the tetramerization of the lactate dehydrogenase subunits may be assessed by any suitable mean available in the state of the art, in particular any suitable biochemical or biophysical method.

Illustratively, biochemical methods, such as, e.g., affinity electrophoresis, bimolecular fluorescence complementation (BiFC), co-immunoprecipitation, tandem affinity purification, intrinsic tryptophan fluorescence, size exclusion chromatography, fractionated centrifugation, cross-linking (SDS PAGE) electrophoresis; or biophysical methods, such as, e.g., biacore, dual polarization interferometry (DPI), dynamic light scattering (DLS), microscale thermophoresis (MST), NMR WaterLOGSY, Saturation Transfer Difference (STD) spectroscopy, Carr Purcell Meiboom Gill (CPMG) pulse sequence and/or static light scattering (SLS), surface plasmon resonance (SPR) may be employed.

In some embodiment, the inhibition of the tetramerization of at least one of the lactate dehydrogenase subunits may be assessed by the ability of the polypeptide of interest to bind to one or more LDH subunit(s) lacking the N-terminus 20 amino acid residues, namely, truncated LDHA or LDHAtr, and truncated LDHB or LDHBtr.

In some embodiment, LDHAtr is represented by an amino acid sequence SEQ ID NO: 3.

In some embodiment, LDHBtr is represented by an amino acid sequence SEQ ID NO: 4.

In some embodiment, when the MST method is implemented, a significant binding of a polypeptide according to the invention to LDHAtr (SEQ ID NO: 3) or LDHBtr (SEQ ID NO: 4), preferably to LDHBtr (SEQ ID NO: 4), may result in a dissociation constant (Kd) comprised from 1 μM to 5 mM, preferably from 50 μM to 3.5 mM.

From 1 μM to 5 mM includes 1 μM, 2 μM, 3 μM, 4 μM, 5 μM, 6 μM, 7 μM, 8 μM, 9 μM, 10 μM, 20 μM, 30 μM, 40 μM, 50 μM, 60 μM, 70 μM, 80 μM, 90 μM, 100 μM, 200 μM, 300 μM, 400 μM, 500 μM, 600 μM, 700 μM, 800 μM, 900 μM, 1 mM, 1.5 mM, 2 mM, 2.5 mM, 3 mM, 3.5 mM, 4 mM, 4.5 mM and 5 mM.

In some aspect, the invention relates to a polypeptide that inhibits the tetramerization of the lactate dehydrogenase subunits, said polypeptide comprising an amino acid sequence of sequence of formula (I)

X1-X2-X3-X4-X5-X6-X7-X8 (I) (SEQ ID NO: 5),

wherein:
X1 represents any amino acid residue, preferentially selected from the group consisting of amino acid residues A, G, K and C;
X2 represents C, T or S;
X3 represents C, L, A, T, cpA (cyclopropyl-L-alanine), chG (L-cyclohexylglycine), chA (cyclohexyl-L-alanine) or mlL (γ-methyl-L-leucine);
X4 represents any amino acid residue, preferentially a positively charged or neutral amino acid residue, preferentially selected from the group consisting of amino acid residues K, C, A and Aib (2-aminoisobutyric acid), and more preferentially amino acid K;
X5 represents any amino acid residue, preferentially a negatively or positively charged or neutral amino acid residue, preferentially selected from the group consisting of amino acid residues E, D, K, A and C, and more preferentially amino acid E;
X6 represents any amino acid residue, preferentially a negatively or positively charged or neutral amino acid residue, preferentially selected from the group consisting of amino acid residues E, K, Q, A, Aib (2-aminoisobutyric acid) and C, and more preferentially amino acid K;
X7 represents C, L, I, cpA (cyclopropyl-L-alanine), chG (L-cyclohexylglycine), chA (cyclohexyl-L-alanine) or mlL (γ-methyl-L-leucine);
X8 represents C, I or G.

In one embodiment, said polypeptide comprises an amino acid sequence of sequence SEQ ID NO: 5 as described above with the proviso that said amino acid sequence SEQ ID NO: 5 has an alpha-helix conformation.

Within the scope of the invention, a “positively charged” amino acid residue is intended to refer to amino acid R, H or K.

Within the scope of the invention, a “negatively charged” amino acid residue is intended to refer to amino acid D or E.

Within the scope of the invention, a “neutral” amino acid residue is intended to refer to amino acid A, V, I, L, M, Q, C, Aib (2-aminoisobutyric acid), S or T.

Within the scope of the invention, “Aib” is intended to refer to 2-aminoisobutyric acid amino acid residue, also referred as to α-aminoisobutyric acid, 2-methylalanine or α-methylalanine.

In some embodiment, the first amino acid residue of the polypeptide is further acetylated. In a particular embodiment, the amino acid residue X1 of SEQ ID NO: 5 is acetylated.

In some embodiment, the last amino acid residue at the C-terminus of the polypeptide is further amidated, so that both Nt and Ct extremities of the polypeptide according to the invention display an NH₂ group. In some embodiments, the last amino acid residue at the C-terminus of the polypeptide is further N-alkyl amidated or N-aryl amidated. In some embodiments, the last amino acid residue at the C-terminus of the polypeptide is further esterified.

In certain embodiments, the —OH group of the free —COOH group of the last amino acid residue at the C-terminus of the polypeptide is replaced by a group selected from an —O-alkyl group, an —O-aryl group, a —NH₂ group, a —N-alkyl amine group, a —N-aryl amine group or a —N-alkyl/aryl group.

Non-limitative examples of suitable alkyl groups include an alkyl in C₁-C₁₂. Non-limitative examples of aryl groups include a phenyl, a tolyl, a xylyl or a naphtyl group, which may be substituted by one or more atom(s) or group(s) from O, N, —OH, —NH₂, a C₁-C₁₂ alkyl group, and a halogen (F, Cl, Br, I). Non-limitative examples of —N-alkyl amine groups include —NR¹R² groups, wherein R¹ and R² represent H or a C₁-C₁₂ alkyl group. Non-limitative examples of a —N-aryl amine group include —NHR³, wherein R³ represents a phenyl, a tolyl, a xylyl or a naphtyl group, which may be substituted by one or more atom(s) or group(s) from O, N, —OH, —NH₂, a C₁-C₁₂, alkyl group, and a halogen (F, Cl, Br, I). Non-limitative examples of —N-alkyl/aryl group include —NR⁴R⁵, wherein R⁴ represents an alkyl in C₁-C₁₂ and wherein R⁵ represents phenyl, a tolyl, a xylyl or a naphtyl group, which may be substituted by one or more atom(s) or group(s) from O, N, —OH, —NH₂, a C₁-C₁₂ alkyl group, and a halogen (F, Cl, Br, I).

In practice, the replacement of the —OH group of the free —COOH group may be performed accordingly to any suitable method known from the state in the art, or a method adapted therefrom.

In some embodiment, amino acid residue L from the amino acid sequence of SEQ ID NO: 5 may be substituted by a non-natural leucine amino acid residue analogue.

Within the scope of the invention, a non-natural leucine amino acid residue analogue is intended to refer to an amino acid residue selected from the group comprising cpA (cyclopropyl-L-alanine), chG (L-cyclohexylglycine), chA (cyclohexyl-L-alanine) and mlL (γ-Methyl-L-leucine).

Because the polypeptide according to the invention has an alpha-helix conformation, the number of amino acid residues known to interfere with said conformation should be limited within the sequence of the polypeptide according to the invention.

Illustratively, amino acid residues such as P and Y are known in the art to unfavor the occurrence of an alpha-helix formation.

In some embodiments, the polypeptide according to the invention comprises at most 3 amino acid residues P and/or Y, at most 2 amino acid residues P and/or Y, at most 1 amino acid residue P and/or Y.

In some embodiment, the polypeptide according to the invention does not comprise any amino acid residue P and/or Y.

In some embodiments, the N-terminal amino acid residue of the polypeptide according to the invention is not amidated.

Means to predict and/or monitor the presence of an α-helix in a peptide of interest are well-known in the art.

Several softwares for predicting the presence of an α-helix in a peptide of interest are available in the art, such as Agadir (Muñoz and Serrano (1994a, b, c, 1997); Lacroix et al. 1998)), PredictProtein (Yachdav et al. (2014)).

In some embodiment, the polypeptide of the invention is a linear polypeptide. In one embodiment, the polypeptide of the invention comprises an amino acid sequence selected from the group consisting of SEQ ID NO: 6 to SEQ ID NO: 22.

In some embodiment, the linear polypeptide according to the invention is selected from the group consisting of SEQ ID NO: 6 (LB19), SEQ ID NO: 7 (LB13), SEQ ID NO: 8 (LB8), SEQ ID NO: 21 (LA19) and SEQ ID NO: 22 (LA8).

In some other embodiment, the polypeptide of the invention is a cyclic polypeptide. In one embodiment, the polypeptide of the invention comprises an amino acid sequence selected from the group consisting of SEQ ID NO: 30 to SEQ ID NO: 35, SEQ ID NO: 55 to SEQ ID NO: 58, SEQ ID NO: 61 to SEQ ID NO: 65, SEQ ID NO: 67 and SEQ ID NO: 68.

In some embodiment, the cyclic polypeptide comprises a CXXXC motif, wherein X represent an amino acid residue conform with the definition of the polypeptide of SEQ ID NO: 5 above.

In some embodiment, both amino acid residues C from the CXXXC motif are alkylated, preferentially by an alkylating agent selected in a group comprising α,α′-bisbromoxylene, hexafluorobenzene, 2,2′-bis(bromomethyl)-1,1′-biphenyl, 1,2-bis(bromomethyl) benzene, 1,4-bis(bromomethyl)benzene, 3,3′-bis(bromomethyl)-1,1′-biphenyl and 4,4′-bis(bromomethyl)-1,1′-biphenyl.

In some embodiments, the cyclic polypeptide is obtained by the mean of a lactam bridge. Within the scope of the invention, the term “lactam bridge” is intended to refer to the covalent binding within the polypeptide of the side-chain of a lysine amino acid residue in order to form an amide bond with the side-chain of a glutamate or an aspartate amino acid residue. Illustratively, lactam bridge formation is disclosed, e.g., in Taylor (2002) and in Aihara et al. (2015).

In certain embodiment, the cyclic polypeptide of the invention is selected from the group consisting of SEQ ID NO: 30 (VS-142-BisAlk), SEQ ID NO: 31 (LT018) and SEQ ID NO: 32 (LT020). In certain embodiments, the cyclic polypeptide of the invention is selected from the group consisting of SEQ ID NO: 55 (MP1), SEQ ID NO: 56 (MP2), SEQ ID NO: 57 (MP3), SEQ ID NO: 58 (MP4), SEQ ID NO: 61 (MP7), SEQ ID NO: 62 (MP8), SEQ ID NO: 63 (MP9), SEQ ID NO:64 (MP10), SEQ ID NO:65 (MP11), SEQ ID NO: 67 (CT-44) and SEQ ID NO: 68 (CT-45).

In some embodiments, said cyclic polypeptide comprises an amino acid sequence selected from the group consisting of SEQ ID NO: 55, SEQ ID NO: 61, SEQ ID NO: 62, SEQ ID NO: 63, SEQ ID NO: 67 and SEQ ID NO: 68.

In some embodiments, the cyclic polypeptide of the invention comprises, or consists in, an amino acid sequence represented by SEQ ID NO: 55 (MP1), i.e., an amino acid sequence represented by CTLKCKLI, wherein the cysteine residues are linked by m-benzyl. In some embodiments, the cyclic polypeptide of the invention comprises, or consists in, an amino acid sequence represented by SEQ ID NO: 61 (MP7), i.e., an amino acid sequence represented by CTLKCKLI, wherein the cysteine residues are linked by p-tetrafluorophenyl. In some embodiments, the cyclic polypeptide of the invention comprises, or consists in, an amino acid sequence represented by SEQ ID NO: 62 (MP8), i.e., an amino acid sequence represented by CTLKCKLI, wherein the cysteine residues are linked by o-benzyl. In some embodiments, the cyclic polypeptide of the invention comprises, or consists in, an amino acid sequence represented by SEQ ID NO: 63 (MP9), i.e., an amino acid sequence represented by CTLKCKLI, wherein the cysteine residues are linked by p-benzyl. In some embodiments, the cyclic polypeptide of the invention comprises, or consists in, an amino acid sequence represented by SEQ ID NO: 67 (CT-44), i.e., an amino acid sequence represented by CT(mlL)KCKLI, wherein the cysteine residues are linked by p-tetrafluorophenyl. In some embodiments, the cyclic polypeptide of the invention comprises, or consists in, an amino acid sequence represented by SEQ ID NO: 68 (CT-45), i e., an amino acid sequence represented by CTLKCK(cpA)I, wherein the cysteine residues are linked by p-tetrafluorophenyl.

In certain embodiments, said cyclic polypeptide comprises an amino acid sequence represented by SEQ ID NO: 61, SEQ ID NO: 67 or SEQ ID NO: 68. In certain embodiments, said cyclic polypeptide comprises an amino acid sequence represented by SEQ ID NO: 61. In certain embodiments, said cyclic polypeptide comprises an amino acid sequence represented by SEQ ID NO: 67. In certain embodiments, said cyclic polypeptide comprises an amino acid sequence represented by SEQ ID NO: 68.

In some embodiment, said lactate dehydrogenase subunit is lactate dehydrogenase B (LDHB) subunit.

In some embodiments, said lactate dehydrogenase subunit is lactate dehydrogenase A (LDHA) subunit.

In a particular embodiment, the polypeptide of the invention is capable of preventing the formation of a functional tetramer of LDHB subunits (corresponding to isoform LDH1) by interacting with the amino acid residues L178, V206, V209, L211 and W227 of a full length LDHB subunit of sequence SEQ ID NO: 2. In a further embodiment, the polypeptide of the invention is also capable to interact with amino acid residues L300 and V303 of a LDHB subunit of sequence SEQ ID NO: 2.

Further, a polypeptide according to the invention is such that it may interact with the amino acid residues L178, V206, V209, L211 and W227 of a LDHB subunit of sequence SEQ ID NO: 2, which form a first alpha-helix, and with the amino acid residues L300 and V303 of a LDHB subunit of sequence SEQ ID NO: 2, which optionally form a second alpha-helix.

The present invention also relates to derivatives of a polypeptide as defined herein.

Indeed, the present invention also encompasses any polypeptide differing from a polypeptide specifically disclosed herein, e.g. a polypeptide of amino acid sequence SEQ ID NO: 5, by one or more substitutions, deletions, additions and/or insertions. Such derivatives may be naturally occurring or may be synthetically generated, for example, by modifying one or more of the above polypeptide sequences of the invention and evaluating one or more inhibiting activities of the polypeptide of the invention and/or using any of a number of techniques well known in the art.

Modifications may be made in the structure of the polypeptides of the present invention and still obtain a functional molecule that encodes a derivative polypeptide with desirable characteristics. When it is desired to alter the amino acid sequence of a polypeptide according to the invention to create an equivalent, or even an improved, variant or portion, one skilled in the art will typically change one or more of the codons of the encoding polynucleotide (e.g., DNA) sequence.

For example, certain amino acid residues may be substituted by other amino acid residues in a protein structure without appreciable loss of its ability to bind other polypeptides (e.g., LDHBtr). Since it is the binding capacity and nature of a protein that defines that protein's biological functional activity, certain amino acid sequence substitutions can be made in a protein sequence, and, of course, its underlying DNA coding sequence, and nevertheless obtain a protein with similar properties.

It is thus contemplated that various changes may be made in the polypeptide sequences of the present invention, or in the corresponding polynucleotide sequences (e.g., DNA sequences) that encode said polypeptides without appreciable loss of their inhibiting activity. In many instances, a variant of a peptide or polypeptide according to the invention will contain one or more conservative substitutions. A “conservative substitution” is one in which an amino acid residue is substituted for another amino acid residue that has similar properties, such that one skilled in the art of peptide chemistry would expect the secondary structure and hydropathic nature of the polypeptide to be substantially unchanged.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include amino acid residues R and K; amino acid residues D and E; amino acid residues S and T; amino acid residues Q and N; and amino acid residues A, V, L and I.

Amino acid substitutions may further be made on the basis of similarity in polarity, charge, solubility, hydrophobicity, hydrophilicity and/or the amphipathic nature of the amino acid residues. For example, negatively charged amino acids include amino acid residues D and E; positively charged amino acids include amino acid residues K and R; and amino acids with uncharged polar head groups having similar hydrophilicity values include amino acid residues A, L, I and V; amino acid residues G and A; amino acid residues N and Q; and amino acid residues S, T, F and Y. Other groups of amino acids that may represent conservative changes include: (1) amino acid residues A, P, G, E, D, Q, N, S, T; (2) amino acid residues C, S, Y, T; (3) amino acid residues V, I, L, M, A, F; (4) amino acid residues K, R, H; and (5) amino acid residues F, Y, W, H.

A derivative of the polypeptide according to the invention may also, or alternatively, contain nonconservative changes. In another embodiment, a derivative differs from a polypeptide sequence by substitution, deletion or addition of five amino acid residues or fewer. Derivatives may also (or alternatively) be modified by, e.g., the deletion or addition of amino acid residues that have minimal influence on the inhibitory capacity of the polypeptide according to the invention.

In another particular embodiment, the polypeptide of the invention comprises whole or part of the tetramerization domain of a lactate dehydrogenase subunit, and more specifically of lactate dehydrogenase A (LDHA) or lactate dehydrogenase B (LDHB) subunit. In said embodiment, the polypeptide comprises the sequence SEQ ID NO: 6, SEQ ID NO: 7, SEQ ID NO: 8, SEQ ID NO: 21 or SEQ ID NO: 22.

In a particular embodiment, the polypeptide of the invention may be represented by the at least 8, preferably at least 9, 10, 11, 12, 13, 14, 15, 16, 17, 18, 19, 20, 21, 22, 23, 24, 25, 50, 75, 100, 125, 150, or 160 amino acid residues of the N-terminus of the lactate dehydrogenase A (LDHA) or lactate dehydrogenase B (LDHB) subunit.

The polypeptide according to the invention does nevertheless not encompass the amino acid sequence of any native lactate dehydrogenase subunit, such as LDHA or LDHB.

In one embodiment, the polypeptide of the invention comprises at least 8, 9, 10, 11, 12, 13, 14, 15, 16, 17, 18 or 19 amino acids.

In one embodiment, the polypeptide of the invention comprises from 8 to 150 amino acids, preferably from 8 to 125 amino acids, more preferably from 8 to 100 amino acids. In one embodiment, the polypeptide of the invention comprises from 8 to 75 amino acids, preferably from 8 to 50 amino acids, from 8 to 40 amino acids, or from 8 to 30 amino acids. In one embodiment, the polypeptide of the invention comprises from 8 to 25 amino acids, from 8 to 20 amino acids or from 8 to 19 amino acids.

In another embodiment, the polypeptide of the invention comprises from 13 to 150 amino acids, preferably from 13 to 125 amino acids, more preferably from 13 to 100 amino acids. In one embodiment, the polypeptide of the invention comprises from 13 to 75 amino acids, preferably from 13 to 50 amino acids, from 13 to 40 amino acids, or from 13 to 30 amino acids. In one embodiment, the polypeptide of the invention comprises from 13 to 25 amino acids, from 13 to 20 amino acids or from 13 to 19 amino acids.

In another embodiment, the polypeptide of the invention comprises from 19 to 150 amino acids, preferably from 19 to 125 amino acids, more preferably from 19 to 100 amino acids. In one embodiment, the polypeptide of the invention comprises from 19 to 75 amino acids, preferably from 19 to 50 amino acids, from 19 to 40 amino acids, or from 19 to 30 amino acids. In one embodiment, the polypeptide of the invention comprises from 19 to 25 amino acids or from 19 to 20 amino acids.

In one embodiment, the polypeptide of the invention comprises at most 100, 90, 80, 70, 60, 50, 40, 30, or 20 amino acids. In a particular embodiment, the polypeptide of the invention comprises at most 19 amino acids.

In one embodiment, the polypeptide of the invention comprises 8, 9, 10, 11, 12, 13, 14, 15, 16, 17, 18 or 19 or more amino acids. In a particular embodiment, the polypeptide of the invention comprises 8 amino acids. In another particular embodiment, the polypeptide of the invention comprises 13 amino acids. In another particular embodiment, the polypeptide of the invention comprises 19 amino acids. In another embodiment, the polypeptide of the invention comprises 20, 21, 22, 23, 24, 25 or more amino acids.

In some embodiments, the amino acid sequence of the polypeptide according to the invention is not SEQ ID NO: 1 or SEQ ID NO: 2.

In a particular embodiment, the polypeptide according to the invention further comprises at least one additional amino acid sequence, hereinafter referred to as a “tag polypeptide”, allowing the said polypeptide of the invention either to be specifically labelled with an epitope for being detected of purified, or allowing the polypeptide of the invention to be targeted to specific cells, a specific tissue or a specific organ, i.e. to a specific body location of the subject. In said embodiment, said polypeptide further comprises at least one tag polypeptide.

Further, in a particular embodiment, the tag polypeptide further allows the polypeptide of the invention to be targeted in the cytoplasm, in the nucleus or in the organelles of target cells, and more preferably of cancer cells.

In a particular embodiment of the invention, the said tag polypeptide is short enough such that it does not interfere with the inhibitory activity of the polypeptide of the invention. Illustratively, suitable tag polypeptides generally have at least six amino acid residues, preferably between about 8 to about 50 amino acid residues, and more preferably, between about 10 to about 20 amino acid residues.

A tag polypeptide for use in the present invention may be such that it provides an epitope to which an anti-tag antibody can selectively bind or it enables the peptide or polypeptide of the invention to be readily purified by affinity purification using an anti-tag antibody or another type of affinity matrix that binds to the epitope tag.

Various tag polypeptides are well known in the art. Examples include poly-histidine (poly-his) or poly-histidine-glycine (poly-his-gly) tags; the flu HA tag polypeptide, the c-myc tag, the Herpes Simplex virus glycoprotein D (gD) tag, the Flag-peptide; the KT3 epitope peptide; an alpha-tubulin epitope peptide; and the T7 gene 10 protein peptide tag.

The polypeptide according to the invention may also be modified so that it can be more easily detected, e.g., by biotinylation or by incorporation of any detectable label known in the art such as radiolabels, fluorescent labels or enzymatic labels. In a particular embodiment, the polypeptide of the invention may thus further comprise any amino acid sequence allowing the said polypeptide to be purified or detected more easily (e.g., a His-Tag, a Biotine tag or a Streptavidine tag).

In a particular embodiment, the polypeptide according to the invention may thus further comprise at least one tag polypeptide consisting in a cell-penetrating peptide (CPPs), also known as protein transduction domain, that facilitates entry into cells. As is well known in the art, cell-penetrating peptides are generally short peptides of up to 30 residues having a net positive charge and act in a receptor-independent and energy-independent manner.

Thus, the polypeptide according to the invention may comprise one or more cell-penetrating peptides. If so, the cell-penetrating peptide may be cleavable inside a cell. Examples of CPPs include those selected in the group consisting of hydrophilic and amphipathic CPPs. Hydrophilic CPPs are peptides composed mainly by hydrophilic amino acids usually rich in amino acid residues R and K.

Examples of hydrophilic CPPs include Antennapedia Penetratin (RQIKWFQNRRMKWKK, SEQ ID NO: 36), TAT (YGRKKRRQRRR, SEQ ID NO: 37), SynB1 (RGGRLSYSRRRFSTSTGR, SEQ ID NO: 38), SynB3 (RRLSYSRRRF SEQ ID NO: 39), PTD-4 (PIRRRKKLRRLK, SEQ ID NO: 40), PTD-5 (RRQRRTSKLMKR SEQ ID NO: 41), FHV Coat-(35-49) (RRRRNRTRRNRRRVR, SEQ ID NO: 42), BMV Gag-(7-25) (KMTRAQRRAAARRNRWTAR, SEQ ID NO: 43), HTLV-II Rex-(4-16) (TRRQRTRRARRNR, SEQ ID NO: 44), D-Tat (GRKKRRQRRRPPQ, SEQ ID NO: 45) and R9-Tat (GRRRRRRRRRPPQ, SEQ ID NO: 46).

Amphipathic CPPs are peptides usually rich in amino acid residue K. Examples of amphipathic CPPs include antimicrobial peptides, such as MAP or transportan: Transportan (GWTLNSAGYLLGKINLKALAALAKKIL, SEQ ID NO: 47), MAP (KLALKLALKLALALKLA, SEQ ID NO: 48), SBP (MGLGLHLLVLAAALQGAWSQPKKKRKV, SEQ ID NO: 49), FBP (GALFLGWLGAAGSTMGAWSQPKKKRKV, SEQ ID NO: 50), MPG (GALFLGFLGAAGSTMGAWSQPKKKRKV, SEQ ID NO: 51), MPG^{(ΔNLS)} (GALFLGFLGAAGSTMGAWSQPKSKRKV, SEQ ID NO: 52), Pep-1 (KETWWETWWTEWSQPKKKRKV, SEQ ID NO: 53), and Pep-2 (KETWFETWFTEWSQPKKKRKV, SEQ ID NO: 54).

The antennapedia-derived penetratin (Derossi et al. (1994)) and the Tat peptide (Vives et al. (1997)), or their derivatives, are in particular widely used tools for the delivery of cargo molecules such as peptides, proteins and oligonucleotides into cells (Fischer et al. (2001)). In another embodiment, the polypeptide of the invention may also comprise a cell-penetrating peptide such as those disclosed in the patent applications WO 2011/157713 and WO 2011/157715 (Hoffmann La Roche®), or derivatives thereof.

In a particular embodiment of the invention, the polypeptide according to the invention is linked to the at least one cell-penetrating peptide (CPPs) by a linker. Within the meaning of the present invention, by “linker”, it is meant a single covalent bond or a moiety comprising series of stable covalent bonds, the moiety often incorporating 1-40 plural valent atoms selected from the group consisting of C, N, O, S and P, that covalently attach a coupling function or a bioactive group to the ligand of the invention. The number of plural valent atoms in a linker may be, for example, 0, 1, 2, 3, 4, 5, 6, 7, 8, 9, 10, 20, 25, or 30 or a larger number up to 40 or more. A linker may be linear or non-linear; and some linkers may have pendant side chains or pendant functional groups (or both).

The polypeptides of the invention may be prepared by methods well known to the skilled person in the art, such as culturing cells transformed or transfected with a vector containing a polynucleotide encoding the desired polypeptide or alternative methods, such as direct peptide synthesis using solid-phase techniques, or in vitro protein synthesis.

The present invention further concerns a polynucleotide encoding a polypeptide according to the invention.

In some embodiments, the polynucleotide comprises a DNA nucleic acid sequence.

The instant disclosure also relates to a nucleic acid vector comprising at least one polynucleotide according to the invention.

Within the scope of the instant invention, the expression “at least one polynucleotide” is intended to include 1, 2, 3, 4, 5, 6, 7, 8, 9, 10, 15, 20, 25, 30, 35, 40, 45, 50 or more polynucleotides.

In some embodiment, the vector allows the controlled expression of said at least one polypeptide.

In certain embodiment, the vector is a viral vector, preferably selected in a group comprising an adenovirus, an adeno-associated virus (AAV), an alphavirus, a herpesvirus, a lentivirus, a non-integrative lentivirus, a retrovirus, vaccinia virus and a baculovirus.

In some embodiments, the polypeptide, the polynucleotide or the nucleic acid vector according to the invention may be comprised in a delivery particle, in particular, in combination with other natural or synthetic compounds, such as, e.g., lipids, protein, peptides, or polymers.

Within the scope of the invention said delivery particle is intended to provide, or “deliver”, the target cells, tissue or organ with the polypeptide, polynucleotide or nucleic acid vector according to the invention.

In some embodiment, the delivery particle may be in the form of a lipoplex, comprising cationic lipids; a lipid nano-emulsion; a solid lipid nanoparticle; a peptide-based particle; a polymer-based particle, in particular comprising natural and/or synthetic polymers; and a mixture thereof.

In some embodiment, a polymer based particle may comprise a synthetic polymer, in particular, a polyethylene imine (PEI), a dendrimer, a poly (DL-Lactide) (PLA), a poly(DL-Lactide-co-glycoside) (PLGA), a polymethacrylate and a polyphosphoesters.

In some embodiment, the delivery particle further comprises at its surface one or more ligand(s) suitable for addressing the polypeptide, the polynucleotide or the nucleic acid vector to a target cell, tissue or organ.

The present invention further concerns a pharmaceutical composition comprising at least one polypeptide, polynucleotide, vector or delivery particle according to the invention, and at least one pharmaceutically acceptable vehicle. In some aspects, the invention relates to a pharmaceutical composition comprising at least one polypeptide according to the invention, and at least one pharmaceutically acceptable vehicle.

In some embodiments, the pharmaceutically acceptable vehicle is selected in a group comprising a solvent, a diluent, a carrier, an excipient, a dispersion medium, a coating, an antibacterial agent, an antifungal agent, an isotonic agent, an absorption delaying agent and combinations thereof. The carrier, diluent, solvent or excipient must be “acceptable” in the sense of being compatible with the polypeptide, or derivative thereof, and not be deleterious to the subject that is administered with it. Typically, the vehicle does not produce an adverse, allergic or other untoward reaction when administered to a subject, preferably a human subject.

For the particular purpose of human administration, the pharmaceutical compositions should meet sterility, pyrogenicity, general safety and purity standards as required by regulatory offices, such as, for example, FDA Office or EMA.

In some embodiments, the carrier may be water or saline (e.g., physiological saline), which will be sterile and pyrogen free. Suitable excipients include mannitol, dextrose, lactose, starch, magnesium stearate, sodium saccharine, cellulose, magnesium carbonate, and the like.

Acceptable carriers, solvents, diluents and excipients for therapeutic use are well known in the pharmaceutical art, and are described, for example, in Remington's Pharmaceutical Sciences, Mack Publishing Co. (A. R. Gennaro ed. 1985). The choice of a suitable pharmaceutical carrier, solvent, excipient or diluent can be made with regard to the intended route of administration and standard pharmaceutical practice. The pharmaceutical compositions may comprise as, or in addition to, the carrier, excipient, solvent or diluent any suitable binder, lubricant, suspending agent, coating agent, or solubilizing agent. Preservatives, stabilizers, dyes and even flavoring agents may be provided in the pharmaceutical composition.

The formulations may conveniently be presented in unit dosage form and may be prepared by any of the methods and the good practices well known in the art of pharmacy. Such methods include the step of bringing into association the peptide or the polypeptide with the carrier which constitutes one or more accessory ingredients.

Formulations in accordance with the present invention that are suitable for oral administration may be presented as discrete units such as capsules, cachets or tablets, each containing a predetermined amount of the polypeptide according to the invention; as a powder or granules; as a solution or a suspension in an aqueous liquid or a non-aqueous liquid; or as an oil-in-water liquid emulsion or a water-in-oil liquid emulsion. The polypeptide of the invention may also be presented as a bolus, electuary or paste.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets. The formulations for use in the present invention may further include other agents conventional in the art having regard to the type of formulation in question, for example those suitable for oral administration may include flavoring agents.

The pharmaceutical composition or medicament of the present invention may be administered orally, parenterally, topically, by inhalation spray, rectally, nasally, buccally, vaginally or via an implanted reservoir. The term administration used herein includes subcutaneous, intravenous, intramuscular, intraocular, intra-articular, intra-synovial, intrasternal, intrathecal, intrahepatic, intralesional and intracranial injection or infusion techniques.

In a preferred embodiment, the pharmaceutical composition or medicament of the present invention is administered parenterally, subcutaneously, intravenously, or via an implanted reservoir.

In one embodiment, the pharmaceutical composition or medicament of the invention is in a form adapted for injection, such as, for example, for intraocular, intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

The treatment may consist of a single dose or a plurality of doses over a period of time. The polypeptide or derivative thereof may be formulated in a sustained release formulation so as to provide sustained release over a prolonged period of time such as over at least 2 or 4 or 6 or 8 weeks. Preferably, the sustained release is provided over at least 4 weeks.

In certain embodiments, the effective amount of the polypeptide to be administered may depend upon a variety of parameters, including the material selected for administration, whether the administration is in single or multiple doses, and the subject's parameters including age, physical conditions, size, weight, gender, and the severity of the disease to be treated.

In certain embodiments, an effective amount of the polypeptide according to the invention may comprise from about 0.001 mg to about 3,000 mg, per dosage unit, preferably from about 0.05 mg to about 1,000 mg, per dosage unit.

Within the scope of the instant invention, from about 0.001 mg to about 3000 mg includes, from about 0.001 mg, 0.002 mg, 0.003 mg, 0.004 mg, 0.005 mg, 0.006 mg, 0.007 mg, 0.008 mg, 0.009 mg, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1 mg, 2 mg, 3 mg, 4 mg, 5 mg, 6 mg, 7 mg, 8 mg, 9 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1,000 mg, 1,100 mg, 1,150 mg, 1,200 mg, 1,250 mg, 1,300 mg, 1,350 mg, 1,400 mg, 1,450 mg, 1,500 mg, 1,550 mg, 1,600 mg, 1,650 mg, 1,700 mg, 1,750 mg, 1,800 mg, 1,850 mg, 1,900 mg, 1,950 mg, 2,000 mg, 2,100 mg, 2,150 mg, 2,200 mg, 2,250 mg, 2,300 mg, 2,350 mg, 2,400 mg, 2,450 mg, 2,500 mg, 2,550 mg, 2,600 mg, 2,650 mg, 2,700 mg, 2,750 mg, 2,800 mg, 2,850 mg, 2,900 mg, 2,950 mg and 3,000 mg per dosage unit.

In certain embodiments, the polypeptide to be administered may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day.

In some particular embodiments, an effective amount of the polynucleotide or nucleic acid vector to be administered may comprise from about 1×10⁵ to about 1×10¹⁵ copies per dosage unit.

Within the scope of the instant invention, from about 1×10⁵ to about 1×10¹⁵ copies includes 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹°, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10′³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴ and 1×10¹⁵ copies, per dosage unit.

The present invention further concerns a medicament comprising at least one polypeptide, polynucleotide, vector or delivery particle according to the invention.

The present invention further concerns a polypeptide, polynucleotide, vector or delivery particle or a pharmaceutical composition according to the invention for use as a medicament. The present invention further concerns a polypeptide, polynucleotide, or a pharmaceutical composition according to the invention for use as a medicament.

In some embodiment, the invention also relates to a polypeptide, polynucleotide, vector or delivery particle or a pharmaceutical composition according to the invention for the manufacture or the preparation of a medicament.

The present invention further concerns a polypeptide, a polynucleotide, a vector, a delivery particle, a pharmaceutical composition, or a medicament according to the invention for use for preventing and/or treating a cancer. The present invention further concerns a polypeptide, a polynucleotide, or a pharmaceutical composition, according to the invention, for use for preventing and/or treating a cancer.

The present invention further relates to a polypeptide, polynucleotide, vector, delivery particle, pharmaceutical composition or medicament according to the invention for use for blocking basal autophagy in a subject in need thereof.

The present invention also relates to a polypeptide, polynucleotide, vector, delivery particle, pharmaceutical composition or medicament according to the invention for use for inhibiting the expansion of cancer cells in a subject in need thereof.

The present invention also concerns a polypeptide, polynucleotide, vector, delivery particle, pharmaceutical composition or medicament according to the invention for use for improving the overall survival of a subject having a cancer.

In some other embodiments, the invention also relates to a method for preventing and/or treating a cancer comprising the step of administering to the subject in need thereof an effective amount of a polypeptide, polynucleotide, vector, delivery particle, pharmaceutical composition or medicament according to the invention.

By “cancer”, as used herein, is encompassed all neoplastic cell growth and proliferation, whether malignant or benign, and all pre-cancerous and cancerous cells and tissues. The terms “cancer” and “cancerous” are intended to refer to or describe the physiological condition in mammals that is typically characterized by unregulated cell growth. Examples of cancer include but are not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulvar cancer, thyroid cancer, hepatic carcinoma and various types of head and neck cancer.

In a particular embodiment, the present invention further concerns a polypeptide, polynucleotide, vector, delivery particle, pharmaceutical composition or medicament according to the invention for preventing and/or treating a cancer involving oxidative cancerous cells and/or glycolytic cancerous cells.

In a particular embodiment of the invention, the subject to be treated is administered a further anticancer therapeutic agent in addition to the polypeptide of the invention, or derivative thereof. For example, when administering the polypeptide to prevent or treat a particular cancer, a further therapeutic agent known to be useful for preventing or treating that cancer may be administered.

Illustratively, when preventing or treating breast cancer, the further therapeutic agent may be an agent known to prevent or treat breast cancer.

Similarly, when preventing or treating uterine cancer, the further therapeutic agent may be an agent known to prevent or treat uterine cancer.

Illustratively, the further therapeutic agent may be any anticancer agent known in the art. Examples of further anticancer therapeutic agent include adriamycin, doxorubicin, epirubicin, 5-fluorouracil, cytosine arabinoside (“Ara-C”), cyclophosphamide, thiotepa, busulfan, cytoxin, taxoids, e.g., paclitaxel (Taxol, Bristol-Myers Squibb Oncology, Princeton, N.J.), and doxetaxel (TaxotereDD, Rhone-Poulenc Rorer, Antony, France), toxotere, methotrexate, cisplatin, melphalan, vinblastine, bleomycin, etoposide, ifosfamide, mitomycin C, mitoxantrone, vincristine, vinorelbine, carboplatin, teniposide, daunomycin, carminomycin, aminopterin, dactinomycin, mitomycins, esperamicins (see U.S. Pat. No. 4,675,187), melphalan and other related nitrogen mustards. Also included in this definition are hormonal agents that act to regulate or inhibit hormone action on tumors such as tamoxifen and onapristone.

It is appreciated that the further therapeutic agent may be administered at the same time as the polypeptide of the invention (i.e. simultaneous administration optionally in a co-formulation) or at a different time to the polypeptide (i.e. sequential administration where the further therapeutic agent is administered before or after the polypeptide is administered). The further therapeutic agent may be administered in the same way as the polypeptide of the invention, or by using the usual administrative routes for that further therapeutic agent.

In a particular embodiment, the polypeptide according to the invention is administered to the subject in need thereof in a therapeutically effective amount.

By “therapeutically effective amount”, it is meant a level or amount of polypeptide, or of a pharmaceutical composition, that is necessary and sufficient for slowing down or stopping the progression, aggravation, or deterioration of one or more symptoms of a cancer; or alleviating the symptoms of a cancer; or curing the cancer, without causing significant negative or adverse side effects to the subject.

In certain embodiments, an effective amount of the polypeptide according to the invention may comprise from about 0.001 mg to about 3,000 mg, per dosage unit, preferably from about 0.05 mg to about 1,000 mg, per dosage unit.

By “subject”, it is meant to refer to a mammal or non-mammal animal, and preferably a human.

In some embodiment, a non-human animal may be selected in a group of valuable economic or pet animals comprising a dog, a cat, a rat, a mouse, a monkey, cattle, a sheep, a goat, a pig and a horse.

In some embodiment, the “subject in need thereof” has been diagnosed as having a cancer and/or metastasis. In one embodiment, the subject is susceptible to develop cancer and/or metastasis. In some embodiment, the “subject in need thereof” is at risk of developing cancer and/or metastasis. In another embodiment, the “subject in need thereof” has already been treated for a cancer and/or metastasis.

The present invention also relates to a method for blocking basal autophagy in a subject in need thereof, comprising the administration to the subject in need thereof an effective amount of a polypeptide or a pharmaceutical composition according to the invention.

The present invention further relates to a method for inhibiting the expansion of cancer cells in a subject in need thereof, comprising the administration to the subject in need thereof an effective amount of a polypeptide or a pharmaceutical composition according to the invention.

In some embodiment, the cancer cells are glycolytic cancer cells. In some alternative embodiment, the cancer cells are oxidative cancer cells.

The present invention further relates to a method for improving the overall survival of a subject having a cancer, comprising the administration to the said subject an effective amount of a polypeptide or a pharmaceutical composition according to the invention.

The present invention also relates to a method for screening a compound affecting the tetramerization of the lactate dehydrogenase subunits comprising the steps of:
a. providing a system comprising truncated lactate dehydrogenase (LDHtr) subunits;
b. providing the system with a candidate compound modulating the activity of a native tetrameric LDH;
c. measuring a level of binding of the candidate compound to a dimer of LDHtr subunits in the presence or in the absence of a polypeptide according to the invention;
   wherein the observation of a competition between the polypeptide and the candidate compound for the binding to the dimer of LDHtr subunits is indicative of the candidate compound being an inhibitor of the tetramerization of the lactate dehydrogenase subunits.

In one embodiment, the observation of a competition between the polypeptide and the candidate compound for the binding to the dimer of LDHtr subunits is indicative of the specificity of the binding of the candidate compound towards the tetramerization site onto the lactate dehydrogenase subunits.

In some embodiments, the LDHtr subunit is a truncated LDHA subunit, in particular a LDHA subunit lacking the tetramerization domain.

In some embodiments, the LDHtr subunit is a truncated LDHB subunit, in particular a LDHB subunit lacking the tetramerization domain.

In some embodiments, the LDHtr subunits comprise both LDHA subunits and LDHB subunits.

In some embodiments, the step of measuring the level of binding of the candidate compound to the dimer of LDHtr subunits may be performed in the presence of an increasing amount of the polypeptide according to the invention.

The present invention also relates to a method for screening a compound affecting the tetramerization of the lactate dehydrogenase subunits comprising the steps of:
a. providing a system comprising truncated lactate dehydrogenase (LDHtr) subunits;
b. providing the system with a candidate compound modulating the activity of a native tetrameric LDH;
c. measuring a level of binding of the candidate compound to a dimer of LDHtr subunits;
   wherein the observation of a binding of the candidate compound to the dimer of LDHtr subunits is indicative of the candidate compound being an inhibitor of the tetramerization of the lactate dehydrogenase subunits.

In some embodiments, the step of measuring a level of binding of a polypeptide according to the present invention, in particular a polypeptide of formula (I) to the LDHtr subunit is performed as a positive control.

In some embodiments, the LDHtr subunit is a truncated LDHA subunit, in particular a LDHA subunit lacking the tetramerization domain.

In some embodiments, the LDHtr subunit is a truncated LDHB subunit, in particular a LDHB subunit lacking the tetramerization domain.

In some embodiments, the LDHtr subunits comprise both LDHA subunits and LDHB subunits.

The present invention also relates to a method for screening a compound affecting the tetramerization of the lactate dehydrogenase subunits comprising the steps of:
a. providing a system (1) comprising truncated lactate dehydrogenase (LDHtr) subunits and a system (2) comprising native tetrameric LDH;
b. providing the systems (1) and (2) with a candidate compound modulating the activity of a native tetrameric LDH;
c. measuring a level of binding (Kd) of the candidate compound to a dimer of LDHtr subunits in system (1) and to a native tetrameric LDH in system (2);
   wherein the observation of a binding of the candidate compound to the dimer of LDHtr subunits in system (1) and wherein the observation of an altered binding of the candidate compound to the native tetrameric LDH in system (2) are indicative of the candidate compound being an inhibitor of the tetramerization of the lactate dehydrogenase subunits, by interacting at the surface of the LDH subunits.

Within the scope of the invention, an altered binding of the candidate compound to the native tetrameric LDH is intended to refer to a level of binding (Kd) of the candidate compound to the native tetrameric LDH that is decreased by at least 50% as compared to the level of binding (Kd) of a said candidate compound to the dimer of LDHtr subunits. As used herein, the term “at least 50%” includes 50%, 60%, 70%, 80%, 90%, 100%, 200%, 250%, 300%, 350%, 400%, 450%, 500%, 600%, 700%, 800%, 900%, 1,000%, 1,500%, 2,000%, 2,500%, 3,000%, 3,500%, 4,000%, 4,500%, 5,000%, 7,500%, 10,000% or more.

The polypeptide, polynucleotide, vector, pharmaceutical composition, delivery particle or medicament of the present invention may be administered orally, parenterally, topically, by inhalation spray, rectally, nasally, buccally, vaginally or via an implanted reservoir. The term administration used herein includes subcutaneous, intravenous, intramuscular, intraocular, intra-articular, intra-synovial, intrasternal, intrathecal, intrahepatic, intralesional and intracranial injection or infusion techniques.

In a preferred embodiment, the polypeptide, polynucleotide, vector, pharmaceutical composition, delivery particle or medicament of the present invention is administered parenterally, subcutaneously, intravenously, or via an implanted reservoir.

In one embodiment, the polypeptide, polynucleotide, vector, pharmaceutical composition, delivery particle or medicament of the invention is in a form adapted for injection, such as, for example, for intraocular, intramuscular, subcutaneous, intradermal, transdermal or intravenous injection or infusion.

The present invention further concerns a kit for preventing and/or treating a cancer comprising at least one polypeptide according to the invention and optionally at least one anticancer agent. The present invention further concerns a kit for preventing and/or treating a cancer comprising at least one polypeptide, a polypeptide or a pharmaceutical composition according to the invention and optionally at least one anticancer agent.

Within the scope of the invention, the expression “at least one anticancer agent” is intended to include 1, 2, 3, 4, 5, 6, 7, 8, 9, 10, anticancer agents, that may be administered in combination with or sequentially to the at least one polypeptide according to the invention.

The instant disclosure also relates to a kit for screening a compound modulating the tetramerization of the lactate dehydrogenase LDHB and/or LDHA subunit(s) comprising:
a LDHtr subunit,
a polypeptide according to the invention.

It is understood that the polypeptide according to the invention may be employed in the later kit as a positive control.

In some embodiments, the LDHtr subunit is a LDHA subunit, in particular a LDHA subunit lacking the tetramerization domain.

In alternative embodiments, the LDHtr subunit is a LDHB subunit, in particular a LDHB subunit lacking the tetramerization domain.

The instant disclosure also relates to a kit for screening a compound modulating the tetramerization of the lactate dehydrogenase LDHB and/or LDHA subunit(s) comprising:
a LDHtr subunit,
a native tetrameric LDH,
a polypeptide according to the invention.

### BRIEF DESCRIPTION OF THE DRAWINGS

FIG. 1A-D is a 3D representation of (A) the full-length LDHB tetramer (PDB code 1I0Z) colored by monomer with the 19 N-terminal amino acids shown by transparency; (B) the 19 N-terminal peptide of one monomer (chain D) superimposed on the trimer formed by the monomers A, B and C, and ((C) and (D)) the main interactions between the 19 N-terminal peptide and the monomers B and C. (Pictures made using Pymol® from Delano Scientific).

FIG. 2A-D is a set of graphs showing the size exclusion chromatograms used to determine the retention volume of (A) full length LDHB and (B) truncated LDHB; (C) superimposition of LDHB and LDHBtr binding assay with their co factor NADH; (D) thermal shift assay of truncated (left) and full length (right) LDHB. Temperature indicated correspond to the thermal shift calculated according to raw fluorescence derivative.

FIG. 3A-C is a set of graphs showing (A) the screening of LB8 analogues at 800 μM against LDHBtr (15 μM) using NMR WaterLOGSY sequence; the dotted line represents an arbitrary threshold of 0.1 corresponding to a 10% increase in NMR WaterLOGSY signal when compared to control experiment; (B) in-silico model of the interacting LB8 with LDHB tetramerization site; (C) the structure-activity relationship of LB8 residues.

FIG. 4A-C is a set of graphs showing (A) a schematic representation of the cysteine cross-linking strategy used to promote helicity; (B) a structure of the best interacting cyclic peptide; (C) Comparison of the binding of this cyclic peptide against full length (up) and truncated LDHB (down).

FIG. 5A-D is a set of graphs showing (A) the fluorescence spectra of full length (LDHBfl) and truncated LDHB (LDHBtr); (B) graph representing the fluorescence spectra of LDH-M in neutral and slightly acidic conditions; (C) graph representing the fluorescence spectra of LDH-M in neutral condition and after renaturation; (D) graph representing the recovery of LDH-M fluorescence intensity over time after renaturation.

FIG. 6A-D is a set of graphs showing the recovery of fluorescence intensity after denaturation with LB8 (A) and LBc (B); (C) tryptophan fluorescence spectra of 1 full length and 2 truncated LDHB; (D) recovery of fluorescence intensity after denaturation with LT018.

FIG. 7 is a graph showing the overall LB19 side chain binding energy (H-bond, Vdw, ionic) calculated from the MOE software using LDHB available X-ray structure (PDB ID 1I0Z). Free energy calculation nicely predicts the overall SAR of LB19 with the 8 N-ter amino acids being the most important for the overall binding.

FIG. 8A-B is a set of graphs showing (A) the MST binding curves of macrocyclic peptide MP7 on dimeric LDHBtr (plod), tetrameric LDH1 (plot 2) and LDH5 (plot 3). Binding curves were extracted from the MST traces at a 10 to 20 s MST on time (n=3) excepted for binding curve with LDH5 which was extracted from the red-dye raw fluorescence (n=3); (B) NanoDSF of various concentrations of human LDH5 exposed to macrocyclic peptide MP7 (n=6). Changes of the 350/330 nm fluorescence emission indicate blue or red shifts and are representative of unfolding events; Plot 1-3: 400 μM MP 7; Plot 1: 300 nM LDH5; Plot 2: 500 nM LDH5; Plot 3: 1,200 nM LDH5; Plot 4: 1,200 nM LDH5.

FIG. 9A-D is a set of graphs showing the impact of MP1 and MP7 on rabbit LDH5 fluorescence recovery after acidic exposure (n=6). (A) 200 nM of rLDH5 renatured in the absence (plot 1) or the presence (plot 2) of 50 μM of MP7; (B) 200 nM of rLDH5 renatured in the absence (plot 1) or the presence (plot 2) of 50 μM of MP1; (C) 200 nM of rLDH5 renatured in the absence (plot 1) or the presence (plot 2) of 50 μM of LB8; (D) 200 nM of rLDH5 renatured in the absence (plot 1) or the presence (plot 2) of 50 μM of LBc.

FIG. 10 is a graph showing the tetrameric state of LDH5 upon increasing concentration of MP7 (plot 1; % Tetrameric) overlayed with the binding curve of MP7 with LDH5 obtained from MST (plot 2; Fraction bound). Tetrameric state is estimated using the 350 nm fluorescence intensity normalized in regard to the spectra of LDH5 (considered as 100% tetrameric) and LDHBtr (considered as 0% tetrameric).

FIG. 11A-E is a set of graphs showing the change in extracellular acidification rate (ECAR) and oxygen consumption rate (OCR) of Mia Paca-2 cells upon addition of macrocyclic peptide MP7 (7) or the control vehicle (Ctrl). (A) basal mitochondrial OCR (in pmol/min/10⁴ cells). (B) maximal mitochondrial OCR (in pmol/min/10⁴ cells). (C) OCR-dependent ATP production. (D) ECAR-linked to glycolysis and (E) the glycolytic capacity ECAR (in mpH/min). N=2, n=15-16. *(p<0.05); ****(p<0.0001).

### EXAMPLES

The present invention is further illustrated by the following examples.

### Example 1

### 1—Experimental Procedures

### 1.1—Peptide Synthesis

All polypeptides employed herein were purchased from GeneCust® (www.genecust.com). The level of purity of peptides was >95%. Structure conformity and purity grade was checked by analytical HPLC analyses and mass spectrometry. All peptides were amidated at their C-terminal unless stated otherwise.

### 1.2—Nuclear Magnetic Resonance (NMR)

Full length human LDHB (LDHB; SEQ ID NO: 2) and truncated LDHB, i.e. a LDHB subunit lacking the first N-terminal 19 amino acid residues (LDHBtr; SEQ ID NO: 4) tagged with a 6His Tag were expressed and purified from E. coli cells as described previously. All experiments were acquired on a Bruker Ascend Avance III 600 MHz equipped with a broadband cryoprobe (Bruker® GmBH, Germany)

### 1.3—NMR WaterLOGSY Experiments

NMR WaterLOGSY was performed on samples prepared in 10% D₂O buffer containing 50 mM sodium phosphate buffer, pH 7.6 and 100 mM NaCl. The concentration of LDH subunits was 15-20 μM. Ligand binding was detected using a NMR WaterLOGSY ephogsygpno.2 advance-version sequence with a is mixing time. Water signal suppression was achieved using excitation sculpting scheme and a 50 ms spinlock was used to suppress protein background signal. For each experiment, 512 scans were collected to yield a 16K points FID. NMR WaterLOGSY intensity was corrected by plotting the intensity difference of the ligand NMR WaterLOGSY spectra recorded in the presence and absence of protein.

For NMR WaterLOGSY screening experiments a correction factor was applied to account for slight concentration variation between samples. To do so, 8 scans 1H NMR spectra with 50 ms spinlock were recorded before NMR WaterLOGSY experiments. The intensity ratio of the aliphatic region (0.700 ppm to 0.955 ppm) with and without protein was used as a correction factor to compare the NMR WaterLOGSY intensity of the polypeptides of interest with and without LDH subunits. An arbitrary threshold of 0.1, corresponding to a 10% decrease in the NMR WaterLOGSY signal intensity between the spectra with and without protein, was set to discriminate between binders and non-binders.

### 1.4-2D Experiments

Polypeptides were dissolved in a 50 mM phosphate buffer pH 7.0 containing 100 mM NaCl, 1 mM TSP and 10% D20. For all experiments, water suppression was achieved using an excitation sculpting scheme. 4 Ktime domain points and 256 increments were applied for all the 2D spectra.

TOCSY experiments were performed using the homonuclear Hartman-Hahn transfer with the dipsi2 sequence with an 80 ms mixing time. 8 scans per spectra, 4 Ktime domain points and 256 increments were recorded.

ROESY experiments were performed using a 2D ROESY sequence with cw spinlock for mixing. 400 ms mixing times were used, and the number of scans taken for was 32.

### 1.5—Size Exclusion Experiments

Size exclusion chromatography was performed using a ÄKTA explorer (GE Healthcare®) equipped with a Superdex 200 Increase 10/300 GL equilibrated with 50 mM sodium phosphate pH 7.6, 100 mM NaCl at 0.7 ml/min. LDHBfl (SEQ ID NO: 2) and LDHBtr (SEQ ID NO: 4) were diluted to 3 μM in assay buffer. The final Injection volume was 100 μl. Prior to experiment, the column was equilibrated for 2× column volume with distillated and filtrated H₂O followed by 3× column volume filtrated buffer. Molecular weight was determined using the Biorad gel filtration standard in the same assay buffer following the manufacturer instructions.

### 1.6—Fluorescence-Based Thermal Shift

Thermal shift assays were performed on a StepOnePlus Real-Time PCR System (Thermo Fisher Scientific®) in 96-well white plates (Roche®). Each well contained 20 μl of 5 μM protein and 5×SYPRO Orange in 50 mM sodium phosphate pH 7.6, 100 mM NaCl. Each plate was sealed with an optically clear foil and centrifuged for 1 min at 1000 rpm before performing the assay. The plates were heated from 20-99° C. at approximately 4° C./min⁻¹. The fluorescence intensity was measured with λex=480 nm and λem=580 nm. The melting temperature (Tm) was obtained by determining the minimum of the first derivative curve of the melt curve.

### 1.7—Microscale Thermophoresis (MST)

MST measurements were performed on a Nanotemper Monolith NT.115 instrument (Nanotemper Technologies®, GmbH) using Red-dye-NHS fluorescent labeling. Each LDH (WT or truncated) sample, purified to homogeneity, was labeled with the Monolith RED-NHS 2^{nd} generation labeling dye according to the supplied protocol (Nanotemper Technologies®, GmbH). Measurements were performed in 50 mM Na-Phosphate pH 7.6 and 100 mM NaCl containing 0.05% Tween-20 in premium treated capillaries (Nanotemper Technologies®, GmbH). The final concentrations of either labeled protein in the assay were 100 nM. The ligands (NADH and peptides) were titrated in 1:1 dilutions following manufacturer's recommendations. All binding reactions were incubated 5′ at room temperature after loading into capillaries. Experiments were performed in triplicates using 40% LED power and medium MST power, LaserOn time was 20 sec, Laser Off time 3 sec. Linear octapeptides were evaluated for their thermophoretic pattern. Longer and cyclic peptide were found to interact with the labelling dye, hence raw fluorescence instead of the thermophoretic pattern was used to extract dissociation constant according to the manufacturer instructions.

### 1.8—Purification of 6His Tagged Human LDH Polypeptides

hLDH (wt and truncated) sequences cloned into pET-28a expression vector was ordered from Genecust®, Luxembourg. The recombinant plasmids were then transformed into host bacterium Escherichia coli Rosetta strain (DE3). The transformants were cultured in LB medium with 50 μg/ml kanamycin and 34 μg/ml chloramphenicol at 37° C. until an optical density of 0.6 was reached. LDHs expression were induced by 1 mM isopropyl-β-D-1-thiogalactopyranoside (IPTG) at 20° C. for 20 h. Then, cells were collected by centrifugation at 5,000 rpm, 4° C. for 25 min. Pellets were suspended into a lysis buffer and then disrupted by sonication, followed by centrifugation at 4° C., 10,000 rpm for 30 min. Insoluble fraction was discarded and 1 μl of β-Mercaptoethanol was added per milliliters of soluble fraction. The purification of recombinant polypeptides was performed using 1 ml His-trap FF-crude columns (GE Healthcare®) according to the instruction of the manufacturer. Finally, concentration was measured using the Bradford method with the Biorad Protein Assay Kit and sample homogeneity was assessed using sulfate-polyacrylamide gel electrophoresis (SDS-PAGE) with Coomassie brilliant blue as staining agent.

### 1.9—Spectrophotometric Experiments

All spectrophotometric experiments were performed with transparent or opaque 96 wells using a spectramax m2e spectrophotometer.

### 1.10—Enzymatic Assay

The dehydrogenase reaction was run in the LDH1 (tetramer of LDHB) physiologically relevant lactate to pyruvate direction following the NADH fluorescence generated during lactate oxidation. The progression of the reaction was monitored as the increase of fluorescence at 340/460 nm.

The Michaelis-Menten constant determination was performed using the GraphPad prism software. Enzymatic reactions were performed in a solution containing phosphate buffer 100 mM at pH 8.3 to enhance lactate to pyruvate oxidation, EDTA 1 mM and DTT 1 mM. Final protein concentration was 7.7 nM for full length LDHB subunit and 13.5 nM for the truncated LDHB subunit (LDHBtr). For NAD⁺Km determination, concentrations of lactate were set to 20 mM while co factor concentrations ranged from 1 μM to 5 mM for full length enzyme and from 50 μM to 10 mM for the truncated form. For lactate Km determination, concentrations of NAD⁺ were set to 1 mM while substrate concentrations ranged from 1 μM to 40 mM for full length enzyme and from 1 mM to 40 mM for the truncated form.

### 1.11—Intrinsic Fluorescence Assay

Following described procedure; rabbit LDHA commercial solution in an ammonium sulfate suspension in (pH-7.0, 3.2 M) was first diluted to 1 mg/ml in a solution of NaCl 200 mM and then dialyzed at 4° C. 2×2 h against 200 mM NaCl. The stock solution of 1 mg/ml was then diluted to 30 μg/ml in NaCl 200 mM to give the assay solution.

Assay solution was mixed 1:1 with either an acetate-chloride buffer (20 mM Acetic acid/Acetate, 180 mM NaCl, pH 5.0) or phosphate buffer (250 mM phosphate pH 7.6). After storage at 4° C. for 30′, samples were removed and diluted 1:1,250 mM phosphate buffer (pH 7.6) to yield a 7.5 μg/ml final concentration of re-associating LDH-M.

Samples of the resulting solutions were then subjected to kinetic experiments of the recovery of intrinsic fluorescence (Exc=286 nm, Em=350 nm, 10′, rt). Full tryptophan fluorescence spectra were afterward recorded (Exc=286 nm, Em=320-400 nm, rt).

### 1.12—Polypeptide Cyclization

The lyophilized crude peptide solution (˜3 mg/mL, —1.5 mM) in NH₄HCO₃ buffer (100 mM, pH=8.0) was treated with TCEP (1.5 equiv) (2.25 μl from 1 M solution in the same NH₄HCO₃ buffer) and stirred for 1 h (700 rpm). The alkylating agent in DMF (˜3 equiv) (100 μl from a 50 mM solution) was added to the solution and shaken for the 2 h (700 rpm). The reaction was quenched by adjusting the pH of the mixture to slightly acidic conditions through the addition of 0.5 N HCl or TFA (150 μl/ml). The crude mixture was then centrifugated at 10,000 rpm 20 minutes. The Supernatant was then analyzed and purified by HPLC/MS.

### 1.13—in Silico Evaluation

Calculation of the free binding energy was performed using the MOE software with the available LDHB (SEQ ID NO: 2) crystallographic structure. No minimization was performed prior to calculation.

### 2—Results

### 2.1—LDH Tetramerization Site in Silico Study

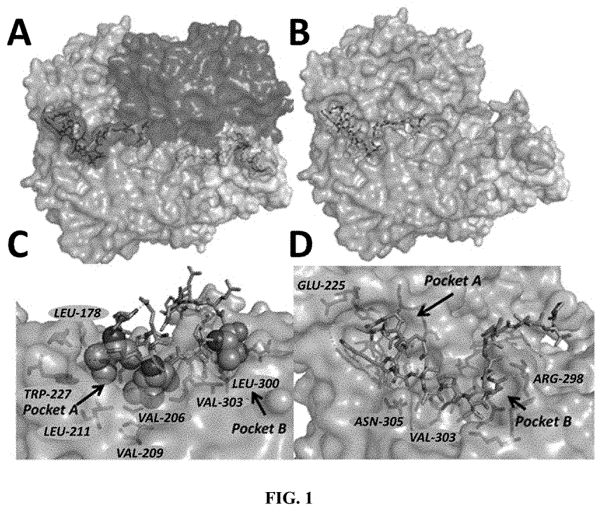

As LDHA and LDHB subunits can hybridize in vitro and in cellulo to give the hetero-tetramers LDH2-3-4, LDHA and LDHB tetramerization site and N-terminal arm are structurally very close. Thus, no selectivity towards one sub-unit is expected to be reached using the considered approach. LDH1 (tetrameric LDHB) and its N-terminal arm were first studied. The analysis of the available LDHB crystallographic structure shows clearly the stabilization of the tetramer by interaction of the 19 N-terminal amino acid polypeptide of each subunit with two other subunits, like four arms embracing the tetramer (FIGS. 1A and 1B). These peptide arms adopt a particular extended conformation with an N-terminal alpha-helix followed by a short β-sheet connected to the subunit through a loop. It should be emphasized that when comparing the four 19 N-terminal amino acids, slight differences regarding the orientations of the side chains can be observed probably because of the peptide flexibility. However, in all cases, the 19 N-terminal amino acid peptide bind to two adjacent pockets (A and B) on two different subunits mainly via non-polar interactions between the amino acid residues L3 and L7, and L178, V206, V209, V211 and W227 in the A-pocket, and V11, and L300 and V303 in the B-pocket (FIGS. 1C and 1D). Polar interactions such as hydrogen bonds (I7 and N305, A9 and V303, A12/E14 and R298) also contribute to the stabilization of the peptide within the A- and B-pocket.

Based on this structural analysis, the pharmacological properties of the LDHB 19 N-terminal amino acid peptide (ATLKEKLIAPVAEEEATVP, namely LB19; SEQ ID NO: 6) on the full length LDH1 enzyme was assessed. Unfortunately, biochemical as well as biophysical evaluation showed no interaction between LB19 and LDH1. It was hypothesized that this lack of effect was stemming from an “unfair” competition between the LDHB N-terminal 19-mer peptide arm and LB19 for the tetramerization site. This led to the design and evaluation of a new protein model allowing for the evaluation of this interaction.

### 2.2—Design and Evaluation of a Dimeric LDH

To address the challenge of evaluating tool compounds at the tetramerization site, a second LDHB truncated of its 19 N-terminal amino acid residues (LDHBtr) was produced. It was hypothesized that this truncated protein, lacking the tetramerization arm, would probably be in a native dimeric state and would therefore allow gaining accessibility to the LDHB tetramerization site.

The recombinant LDHBtr (SEQ ID NO: 4) form was produced in E. coli and was shown to be in a native dimeric state by size exclusion chromatography (SEC), diffusion light scattering (DLS) and intrinsic fluorescence. Furthermore, the affinity of rLDHBtr for its cofactor was evaluated using microscale thermophoresis (MST) and a Kd value of 21 μM+/−5 μM was obtained, similarly to the one of the full length LDHB (Kd=24 μM+/−8 μM), thus indicating a correct fold of the protein “Rossman domain”. The catalytic properties of rLDHBtr were also evaluated using standard biochemical assay (FIG. 1) and showed very weak activity with compared to the full-length LDHB with a 5-fold increase in Michaelis-Menten constant Km as well as a 10-fold decrease in maximal velocity Vmax for both substrate and cofactor (Table 1).

TABLE 1
Enzymatic characteristics of full length and truncated LDHB subunits

| | LDHBtr | LDHBfl |
|---|---|---|
| Km NAD⁺ (mM) | 0.578 | 0.153 |
| Km Lactate (mM) | 6.52 | 33.77 |
| Vmax NAD⁺ (μM/min) | 0.29 | 3.33 |
| Vmax Lactate (μM/min) | 1.11 | 11.41 |

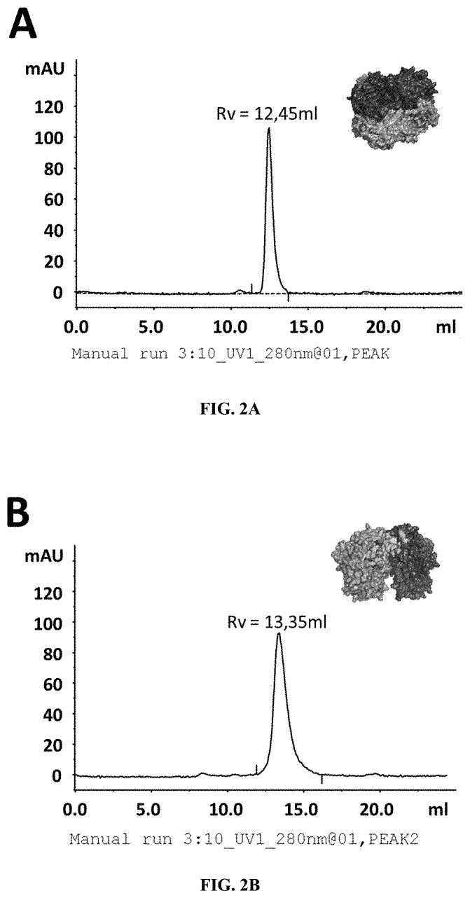

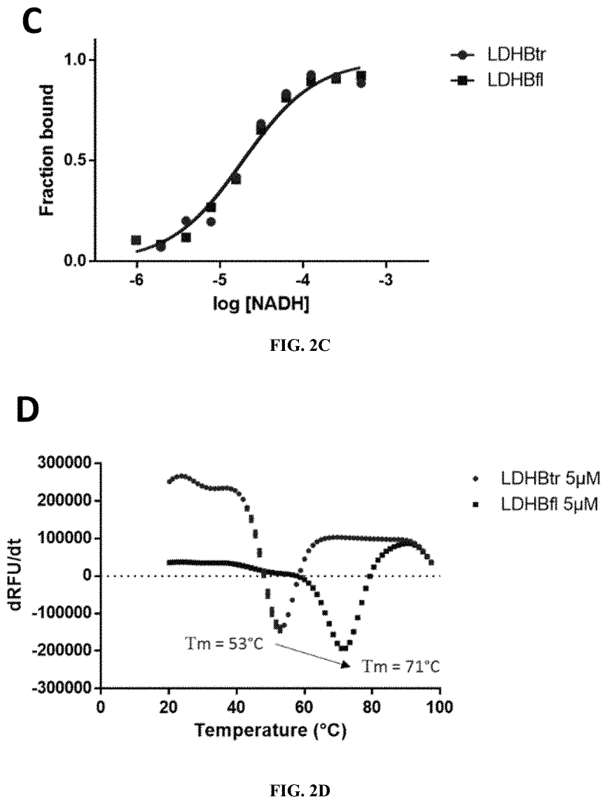

Finally, LDHBtr stability was evaluated using thermal shift assay and TYCHO NT.6. The dimeric LDHB was found to be deeply destabilized when compared to the tetrameric LDHB with 18° C. and 24° C. shift in melting temperature, respectively (FIG. 2). Conclusively, these results indicate that the truncated LDHB is a well folded, but poorly active dimeric protein. It besides demonstrates that targeting LDH tetramerization could destabilize the enzyme as well as weaken its activity.

### 2.3—Study and Optimization of the Interaction Between LB19 and LDHBtr Tetramerization Site

Biophysical evaluation of the interaction between LDHBtr (SEQ ID NO: 4) and LB19 (SEQ ID NO: 6) was performed using two biophysical orthogonal methods: NMR WaterLOGSY and Microscale thermophoresis (MST). According to MST analysis, LB19 interacts with LDHBtr with a Kd of 270 μM [+/−70 μM]. NMR WaterLOGSY analysis showed a positive signal stemming from an interaction between LB19 and LDHBtr. Analysis of NMR WaterLOGSY spectra allowed for an epitope mapping of the interaction between the two molecules. Interestingly, LB19 N-terminal residues underwent more saturation transfer than their C terminal counterpart, meaning that LB19 N-terminal residues could account for most of the binding strength. Accordingly, calculation of the overall binding energy of LDHB native arm showed similar results.

Following these observations, some C-terminal amino acids were removed from LB19 to keep only those from the N-terminal end accounting for the binding with LDHBtr. This led to evaluate the binding of the polypeptide LB13 (SEQ ID NO: 7) to LDHBtr (SEQ ID NO: 4). In accordance with the previous results, LB13 resumed all the interacting residues and thus presented the exact same NMR WaterLOGSY spectrum than LB19. Moreover, MST analysis confirmed that the interaction was only slightly weakened with a Kd=605 μM [+/−290 μM]. Further size reduction of LB13 led to the evaluation of LB8 (ATLKEKLI; SEQ ID NO: 8), which apart from a valine residue summarized the same interaction residues as in LB13. Again, apart from a small drop in the interaction strength (Kd=1.4 mM [+/−0.4 mM]), LB8 exactly matched with the N-terminal alpha-helix of LDHB N-terminal arm and therefore can be anticipated as a “Hot-spot” of the interaction between LDHB tetramerization site and its N-terminal arm. A LB19 central fragment (LIAPVAE, namely LBc; SEQ ID NO: 26) was also evaluated as a negative control and found not to demonstrate any appreciable saturation transfer under these conditions.

### 2.4—LB8 SAR

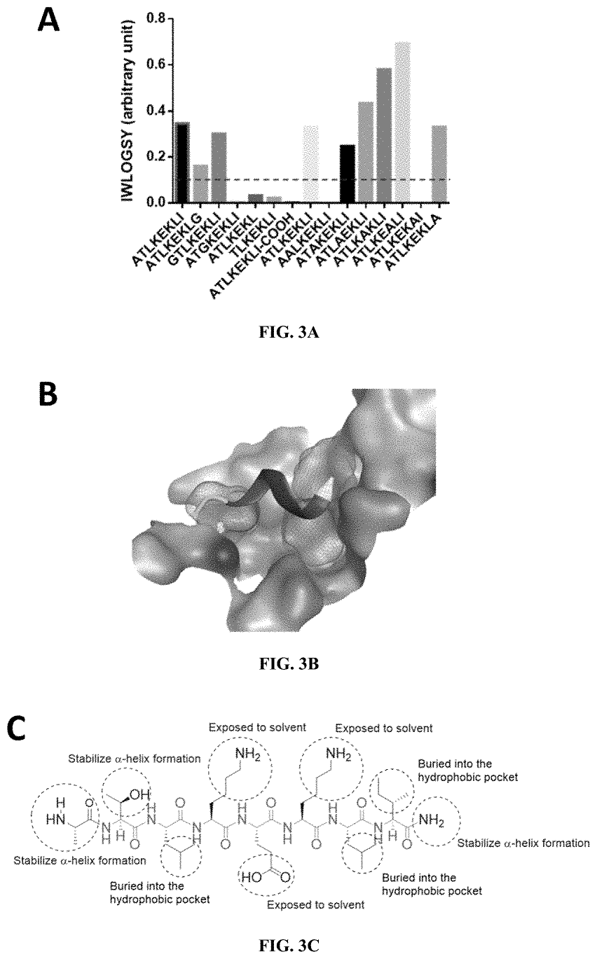

The evaluation of the structure-activity relationship between LB8 (SEQ ID NO: 8) and LDHB tetramerization site was further examined. As the active conformation of LB8 was expected to be an a-helix, a combination of in silico and experimental evaluation was used to unravel LB8 SARs. A set of 15 LB8 structural analogues was constructed and further analyzed by NMR WaterLOGSY experiments at a single concentration of 800 μM to identify structural modifications that would result in a loss of saturation transfer (Table 2 and FIG. 3). Taken together, these results allowed to get insights into LB8 structure-activity relationships.

TABLE 2
Binding properties of linear polypeptides according to the invention
Linear peptides

| | | Binding | |
|---|---|---|---|
| | | using | |
| | | WaterLOGS | Kd using MST |
| Name | Sequence | Y (800 μM) | experiment |
| LB19 | ATLKEKLIAPVAEEEATVP | + | 270 μM +/− 70 μM |
| (SEQ ID NO: 6) | | | |
| LB13 | ATLKEKLIAPVAE | + | 605 μM +/− 290 μM |
| (SEQ ID NO: 7) | | | |
| LA19 | ATLKDQLIYNLLKEEQTPQ | + | N.D |
| (SEQ ID NO: 21) | | | |
| LB8 | ATLKEKLI | + | 1.44 mM +/− 0.4 mM |
| (SEQ ID NO: 8) | | | |
| LA8 | ATLKDQLI | + | 3.1 mM +/− 1.1 mM |
| (SEQ ID NO: 22) | | | |
| LB8-A1 | AALKEKLI | − | 8.2 mM +/− 5.1 mM |
| (SEQ ID NO: 23) | | | |
| LB8-A2 | ATAKEKLI | + | N.D |
| (SEQ ID NO: 9) | | | |
| LB8-A3 | ATLAEKLI | + | N.D |
| (SEQ ID NO: 10) | | | |
| LB8-A4 | ATLKAKLI | + | N.D |
| (SEQ ID NO: 11) | | | |
| LB8-A5 | ATLKEALI | + | N.D |
| (SEQ ID NO: 12) | | | |
| LB8-A6 | ATLKEKAI | − | >10 mM |
| (SEQ ID NO: 24) | | | |
| LB8-A7 | ATLKEKLA | + | N.D |
| (SEQ ID NO: 13) | | | |
| LB8-AL7 | ATLKEKL | − | N.D |
| (SEQ ID NO: 25) | | | |
| Ac-LB8 | Ac-ATLKEKLI | + | N.D |
| (SEQ ID NO: 14) | | | |
| LBc | Ac-LIAPVAE-NH2 | − | N.D |
| (SEQ ID NO: 26) | | | |
| LB8-AcTI7 | Ac-TLKEKLI | − | N.D |
| (SEQ ID NO: 27) | | | |
| LB8-T17 | TLKEKLI | − | N.D |
| (SEQ ID NO: 28) | | | |
| LB8-A3-A5 | ATLAEALI | + | N.D |
| (SEQ ID NO: 15) | | | |
| LB8-G3 | ATGKEKLI | − | >10 mM |
| (SEQ ID NO: 29) | | | |
| LB8-G8 | ATLKEKLG | + | N.D |
| (SEQ ID NO: 17) | | | |
| LB8-G1 | GTLKEKLI | + | N.D |
| (SEQ ID NO: 16) | | | |
| LB8-Aib1 | ATL(Aib)EKLI | + | N.D |
| (SEQ ID NO: 18) | | | |
| LB8-Aib2 | ATLKE(Aib)LI | + | N.D |
| (SEQ ID NO: 19) | | | |
| LB8-Aib3 | ATL(Aib)E(Aib)LI | + | N.D |
| (SEQ ID NO: 20) | | | |

In agreement with the analysis of the crystallographic data, two L amino acid residues as well as the C-terminal isoleucine were found to be required for the binding. The in silico model extracted from the LDHB 3D structure indicated that these aliphatic side chains projected towards hydrophobic cavities at the tetramerization site. NMR WaterLOGSY mapping of the saturation transfer intensity confirmed that lipophilic residues undergo more saturation transfer that any other, thus interacting more closely at the tetramerization site.

In LB8 (SEQ ID NO: 8), amino acid residue switch T to A, as well as a removal of any of the terminal residues, also resulted in abrogation of the interaction (Table 2 and FIG. 3A). Based on this in silico model as well as on Agadir helicity calculation, it was hypothesized that these modifications would destabilize the active alpha-helix conformation. Modifications of other side chains residues had no impact over the peptide interaction with LDHB tetramerization site.

### 2.5—Cyclization

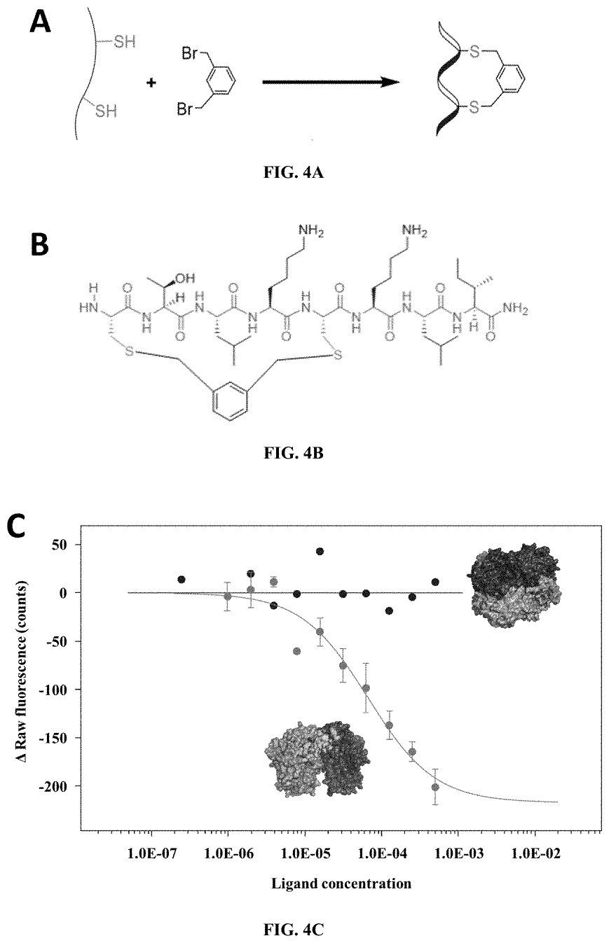

It was expected that LB8 weak binding could be accounted for its poor helical propensity that would result in a huge entropy cost prior to binding. Indeed, 2D NOESY and ROESY analysis confirmed the absence of the alpha-helix characteristic cross coupling in the N-terminus region. Moreover, previous studies have shown entropy-mediated gain in potency by constraining the conformational freedom of peptides. LB 8 side chain to side chain cyclization was hence performed to promote its helicity. Many strategies are described for peptide macrocyclization (Hill et al. (2014)). Among them, cysteine alkylation with an alpha-helix promoting agent already demonstrated strong results in enhancing small peptides helicity, and hence affinity (FIG. 4A) (Jo et al. (2012)). Based on LB8 SAR we therefore introduced cysteine at various i and i+4 position and alkylated these peptides using α,α′ bisbromoxylene. The resulting cyclic peptides were then assayed for their binding using NMR WaterLOGSY and MST experiments in an orthogonal way.

TABLE 3
Binding properties of cyclic peptides according to the invention
Cyclic peptides

| | | Binding using | |
|---|---|---|---|
| | | WaterLOGSY | Kd using MST |
| Name | Sequence | (800 μM) | experiment |
| VS-142-BisAlk | ACLKECLI | + | 233 μM +/− 113 μM |
| (SEQ ID NO: 30) | | | |
| LT018 (SEQ ID NO: 31) | CTLKCKLI | + | 66 μM +/− 32 μM |
| LT020 (SEQ ID NO: 32) | ATLKCKLIC | N.D | 477 μM +/− 116 μM |
| LT021 (SEQ ID NO: 33) | ACTLKCKLI | + | |
| LT022 (SEQ ID NO: 34) | CATLCEKLI | | |
| CB-09 (SEQ ID NO: 35) | ATCKEKCI | | |

Among them, the LT018 polypeptide (SEQ ID NO: 31) revealed to be the most promising one with an apparent 30-fold increase in potency (Kd=66 μM+/−32 μM) compared to LB8 (SEQ ID NO: 8) and an intense saturation transfer. However, despite this increased affinity, LT018 polypeptide (SEQ ID NO: 31) was still not able to compete with LDHB native arm (FIG. 4). It nevertheless constituted a promising tool for further LDH tetramerization site evaluation.

### 2.6—VS-142-BisAlk Polypeptide Inhibits LDH Tetramerization

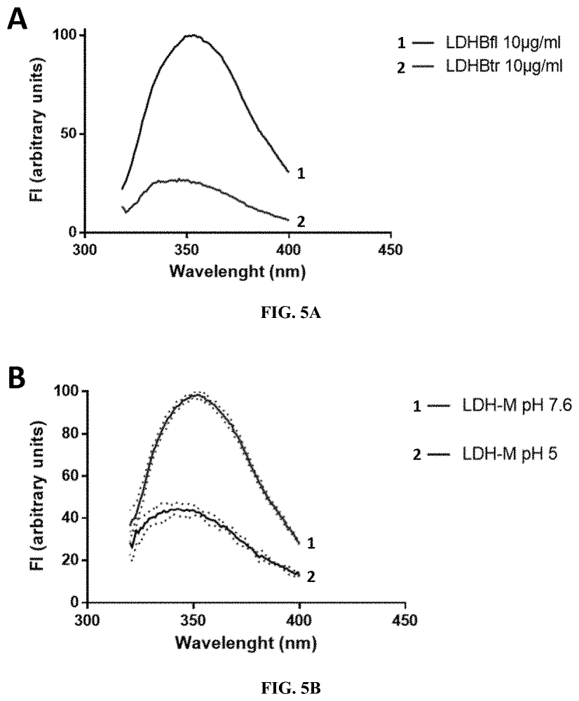

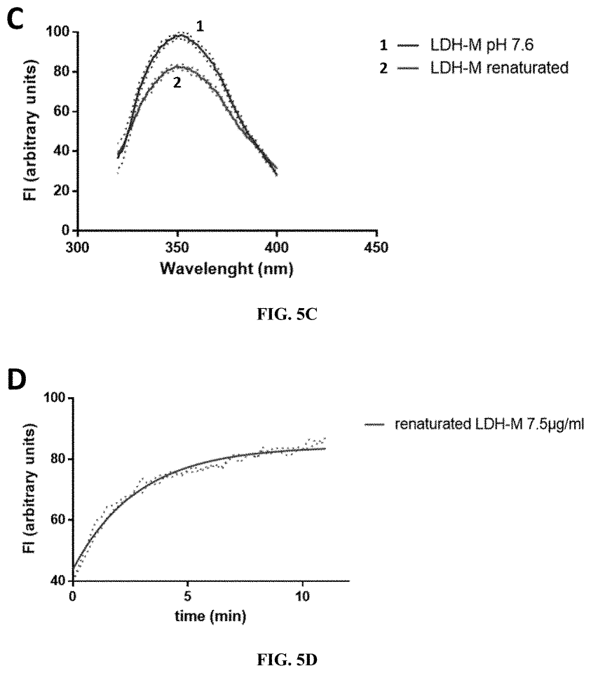

Following the observation that LT018 polypeptide (SEQ ID NO: 31) was not able to compete with LDHB native arm and thus was not able to disrupt an already formed LDHB tetramer, it was reasoned that it could maybe bind to the tetramerization site in a pre-dissociation dependent manner. To confirm this hypothesis, experiments were therefore designed to follow the recovery of the LDH tetrameric form after a pre-dissociation initiated in slightly acidic conditions. Briefly, six tryptophan residues are found in the LDH structure, three of them being located at the dimer-dimer interface. As the tryptophan quantum yield decreases in polar environment, dimeric LDHs show very weak tryptophan fluorescence compared to tetrameric one. Accordingly, in acidic conditions (pH 5.0) LDH shows a decrease in tryptophan fluorescence that is correlated to the dissociation of the tetramer (Rudolph and Jaenicke (1976); FIG. 5). The recovery of fluorescence upon pH neutralization is therefore a direct measure of the tetramer re-association.

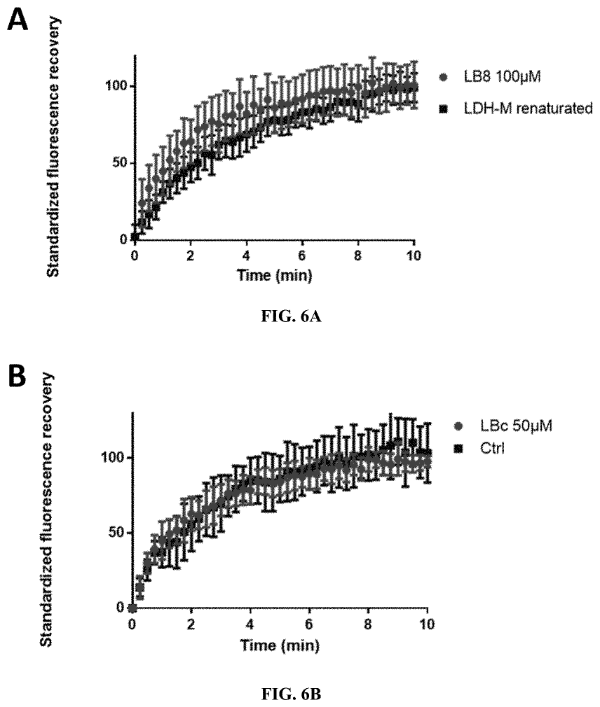

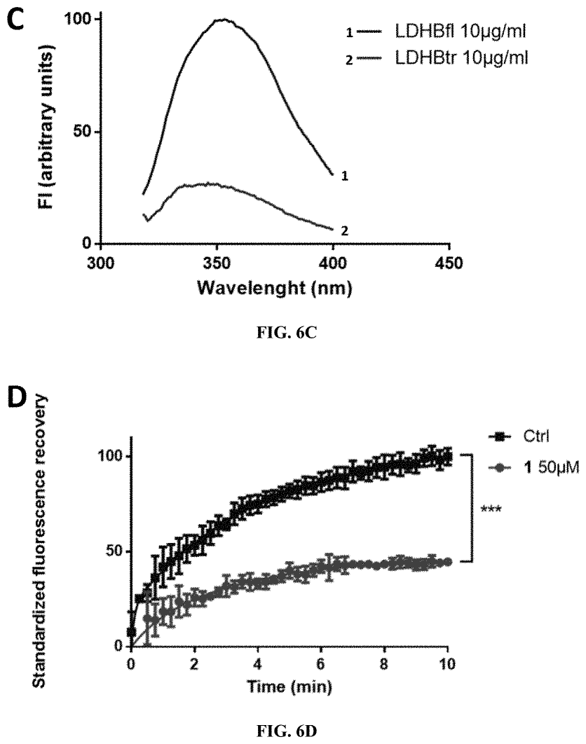

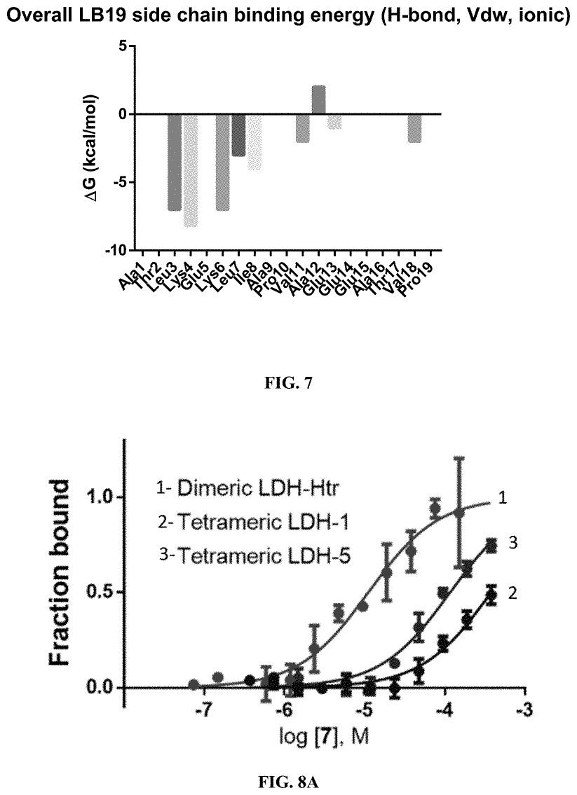

Strikingly, the LT018 polypeptide (SEQ ID NO: 31) nicely interfered with the fluorescence recovery at 50 μM (FIG. 6D) while LB8 had no effects up to 100 μM (FIG. 6A). LBc (SEQ ID NO: 26) was also used as negative control and had no effect upon LDH re-association (FIG. 6B). Conclusively, these results demonstrate that LT018 polypeptide (SEQ ID NO: 31) can interfere with the LDH tetramerization process.

### Example 2

### 1—Materials and Methods

### 1.1—Chemicals and Peptides

All reagents were purchased from chemical suppliers and used without purification. Rabbit and recombinant human LDHA were purchased respectively from Sigma-Aldrich® and Abnova®. Linear peptides used directly in biophysical experiments were purchased from Genecust® and linear peptides used for cysteine stapling were synthetized by solid-phase peptide synthesis. Lactam cyclic peptides were purchased from Proteogenixa Structure conformity and purity grade (>95%) were assessed by analytical high-performance liquid chromatography (HPLC) analysis and mass spectrometry (MS) for both commercial and synthetized peptides. All peptides were amidated at their C-termini

### 1.2—Peptide Synthesis

All peptides used for cysteine cross-linking procedures were synthetized on a 0.05 or 0.1 mmol scale using a Rink amide AM resin (Bachem®) (substitution 0.5-1.2 mmol/g). Fluorenylmethyloxycarbonyl (Fmoc)-protected amino acids (5-fold excess) were activated with 1 equivalent of hexafluorophosphate benzotriazole tetramethyl uronium (HBTU) and 2 equivalents of diisopropylethanolamine (DIPEA) (equivalent relative to the amino acid). Coupling was performed in N-methyl-2-pyrrolidone (NMP) for 60 min at room temperature. Fmoc deprotection was carried out using 20% piperidine in NMP for 10 min at room temperature. Side chain deprotection as well as simultaneous cleavage from the resin were achieved using a mixture of Trifluoroacetic acid (TFA)/Triisopropylsilane/water/thioanisole (90/2.5/2.5/5) at room temperature for 2 h. TFA was then evaporated under nitrogen flux, and the crude peptide was precipitated using ice cold diethyl ether. Crude peptides were then analyzed using an Agilent® (1100 series) HPLC single quadrupole (InfinityLab, ESI+) system equipped with a kinetex 5 μm EVO C18 (150×4.6 mm), and subsequently lyophilized for further use.

### 1.3—Synthesis of the Cross-Linked Peptides

Stapling using hexafluorobenzene was performed by following the procedure described by Spokoyny et al. (2013). To a lyophilized sample of peptide (˜7.5 μmoles) was added 1.9 mL of 100 mM solution (— 25 equiv.) of hexafluorobenzene in DMF and 1.5 mL of 50 mM solution of tris base in DMF. Solution was left under agitation at room temperature for 5 h. Resulting mixture was diluted with 2 times volume of 0.1% TFA solution in water and subjected to analysis and purification on HPLC as described above.

### 1.4—Microscale Thermophoresis (MST)

MST measurements were performed on a Nanotemper Monolith NT.115 instrument (NanoTemper Technologies®) using Red-dye-NHS fluorescent labeling. Each LDH sample, purified to homogeneity, was labeled with the Monolith Red-dye-NHS 2^{nd} generation labeling dye (NanoTemper Technologies®), according to the manufacturer's instructions. Measurements were performed in 50 mM sodium phosphate, pH 7.6, and 100 mM NaCl containing 0.05% Tween-20 in premium-treated capillaries (NanoTemper Technologies®). The final concentrations of either labeled protein in the assay were 100 nM. The ligands (NADH and peptides) were titrated in 1:1 dilutions following manufacturer's recommendations. All binding reactions were incubated for 5 min at room temperature after loading into capillaries. Experiments were performed in triplicates using 40% LED power, medium MST power, Laser On time 20 s and Laser Off time 3 s. Peptides were evaluated for their thermophoretic pattern, and Kd's were extracted from raw data at a 10 to 20 s MST on time according to manufacturer's instructions. Regarding interaction of 7 with LDH5, Kd was extracted from the raw fluorescence. A denaturation test was performed accordingly to manufacturer recommendation and excluded any nonspecific spectral interaction between 7 and the red-dye. All Kd's of interacting macrocycles and peptides were obtained in triplicate and corrected by taking into account the molecular weight of the TFA counter ion. Peptide ATGKEKLI (LB8-G3; SEQ ID NO: 29) was used as a negative control, and displayed no appreciable binding when compared to LB8 (SEQ ID NO: 8).

### 1.5—Spectrophotometric Experiments

All spectrophotometric experiments were performed with opaque 96-well plates using a Spectramax m2e spectrophotometer (Molecular Devices).

### a) Kinetic Assays

The dehydrogenase reaction was run in the LDH1 physiologically relevant lactate to pyruvate direction following the NADH fluorescence generated during lactate oxidation to pyruvate. The progression of the reaction was monitored as the increase of fluorescence at 340/460 nm. The Michaelis-Menten Km constant determination was performed using the GraphPad prism 7.0 software. Enzymatic reactions were performed in a solution containing phosphate buffer 100 mM at pH 8.0 to enhance lactate to pyruvate oxidation, EDTA 1 mM. Final protein concentration was 7.7 nM for LDHB and 13.5 nM for LDHBtr. For NAD+ Km determination, concentrations of lactate were set to 20 mM for LDHB and 150 mM for LDHBtr, while cofactor concentrations ranged from 1 μM to 5 mM for LDHB and from 50 μM to 10 mM for LDHBtr. For lactate Km determination, concentrations of NAD+ were set to 1 mM while substrate concentrations ranged from 1 μM to 30 mM for LDHB and from 1 mM to 40 mM for LDHBtr.

### b) Intrinsic Fluorescence Assays

Full tryptophan fluorescence spectra were recorded using an excitation wavelength of 286 nm and recording the emission spectra from 320 nm to 400 nm at room temperature. Raw fluorescence of every experiments was further subtracted to a corresponding control experiment without the protein. Experiments were performed in a 50 mM sodium phosphate and 100 mM NaCl, pH 7.6, buffer. For LDH dissociation into subunits, increasing amounts of guanidinium/HCl were put in contact with the studied proteins (1.3 μM), and fluorescence spectra were recorded afterwards. Guanidinium/HCl concentrations ranged from 0.3 M to 2 M.

### c) Denaturation Assays

Rabbit LDHA commercial solution (Sigma-Aldrich®) in an ammonium sulfate suspension (pH-7, 3.2 M) was first diluted to 1 mg/ml in a solution of NaCl 200 mM and then dialyzed at 4° C. 2×2 h against 200 mM NaCl. The stock solution of 1 mg/ml was then diluted to 30 μg/ml (800 nM) in NaCl 20 0 mM to give the assay solution. Assay solution was mixed 1:1 with acetate-chloride buffer (20 mM Acetic acid/Acetate, 180 mM NaCl, 1 mM DTT pH 5) and stored on ice for 30 minutes. Samples were then removed from ice and let warm up for 2 minutes. The acidic solution was then diluted 1:1 with a 250 mM phosphate buffer (pH 7.6) containing or not the inhibitory peptide to yield a 7.5 μg/ml (200 nM) final concentration of re-associating LDHA. Samples of the resulting solutions were then subjected to kinetic experiments of the recovery of intrinsic fluorescence (Exc=286 nm, Em=350 nm, 10′, rt).

### 1.6—Statistics

All quantitative data are expressed as means±SEM. Error bars are sometimes smaller than symbols. n refers to the total number of replicates per group. All experiments were repeated at least twice independently. Data were analyzed using the GraphPad Prism 7.0 software. Student's t test, one-way ANOVA and two-way ANOVA were used where appropriate. P<0.05 was considered to be statistically significant.

### 2—Results

### 2.1—Binding of Macrocyclic Peptides (MP) to Truncated LDHB (LDHBtr)

Following identification of the optimal i and i+4 position for LB8 polypeptide cyclization, macrocyclic peptides (MP) bearing other linkers (see Table 4) we investigated, including p-tetrafluorophenyl (MP7), o-benzyl (MP8), p-benzyl (MP9), as well as a lysine to aspartate lactam bridge between the side-chains of the K₁ and D₅ residues (MP10).

TABLE 4
Code, structure, dissociation constants (IQ) and 95% confidence
interval of evaluated macrocycles against truncated LDHB

| Name | Sequence | Linker | K_{d}* | | CI_{95%} | |
|---|---|---|---|---|---|---|
| LB8 (SEQ ID NO: 8) | ATLKEKLI | — | 1.05 | mM | 0.55 to 2.01 | mM |
| MP1 (SEQ ID NO: | CTLKCKLI | m-benzyl | 64 | μM | 55 to 75 | μM |
| 55) | | | | | | |
| MP2 (SEQ ID NO: | ACTLKCKLI | m-benzyl | 67 | μM | 55 to 82 | μM |
| 56) | | | | | | |
| MP3 (SEQ ID NO: | ACLKECLI | m-benzyl | 787 | μM | 529 to 1171 | μM |
| 57) | | | | | | |
| MP4 (SEQ ID NO: | ATLKCKLIC | m-benzyl | 398 | μM | 246 to 645 | μM |
| 58) | | | | | | |
| MP5 (SEQ ID NO: | ATLKECLIAC | m-benzyl | >>1 | mM | ND | |
| 59) | | | | | | |
| MP6 (SEQ ID NO: | CATLCEKLI | m-benzyl | >>1 | mM | ND | |
| 60) | | | | | | |
| MP7 (SEQ ID NO: | CTLKCKLI | P- | 113 | μM | 9 to 14 | μM |
| 61) | | tetrafluorophenyl | | | | |
| MP8 (SEQ ID NO: | CTLKCKLI | o-benzyl | 25 | μM | 21 to 29 | μM |
| 62) | | | | | | |
| MP9 (SEQ ID NO: | CTLKCKLI | p-benzyl | 11 | μM | 98 to 131 | μM |
| 63) | | | | | | |
| MP10 | Ac-KTLKDKLI | Lactam bridge K₁- | 142 | μM | 117 to 174 | μM |
| (SEQ ID NO: 64) | | D₅ | | | | |
| MP11 | ATLKEKLI | Lactam bridge | 465 | μM | 355 to 607 | μM |
| (SEQ ID NO: 65) | | Nter-E₅ | | | | |
| MP12 | ATLKEKLI | Lactam bridge K₆- | >>1 | mM | ND | |
| (SEQ ID NO: 66) | | Cter | | | | |
| CT-44 | CT(m1L)KCKLI¹ | p- | 9.43 | μM | 7.30 to 12.17 | μM |
| (SEQ ID NO : 67) | | tetrafluorophenyl | | | | |
| CT-45 | CTLKCK(cpA)I² | p- | 7.82 | μM | 6.13 to 9.96 | μM |
| (SEQ ID NO : 68) | | tetrafluorophenyl | | | | |

*K_{d} were extracted from MST traces at 10 s to 20 s on time (n = 3 for macrocyclic peptides MP1-MP4 and MP7-MP11, n = 2 for macrocyclic peptides MPS-MP6 and MP12).
ND, not determined.
¹mlL represents γ-methyl-L-leucine.
²cpA represents cyclopropyl-L-alanine.

Strikingly, Kd evaluation of these macrocyclic peptides revealed an impact of the overall constrain imposed by the linker on the evaluated affinity. Indeed, p-tetrafluorophenyl (MP7; SEQ ID NO: 61) and o-benzyl (MP8; SEQ ID NO: 62) analogues yielded a supplementary 2-fold to 6-fold improvement in affinity when compared to macrocyclic peptide MP1 (SEQ ID NO: 55), with K_{d}'s of 11 μM and 25 μM, respectively. Comparatively, less constraining linkers, p-benzyl (MP9; SEQ ID NO: 63) and the Ki-D5 lactam bridge (MP10; SEQ ID NO: 64), resulted in weakly potent derivatives with K_{d}'s of 113 μM and 142 μM, respectively. As compared to macrocyclic peptide MP7 (SEQ ID NO: 61), substitution of leucine in amino acid position 3 with γ-methyl-L-leucine, as in macrocyclic peptide CT-44 (SEQ ID NO: 67) did not affect the binding properties. Similarly, substitution of leucine in amino acid position 7 with cyclopropyl-L-alanine, as in macrocyclic peptide CT-45 (SEQ ID NO: 45), resulted in an unaltered K_{d} value, or even a slightly improved K_{d} value.

The influence of a lactam bridge between the N-terminal amino group and the carboxylic acid on the side-chain of the E₅ residue was further investigated, as these two moieties can be found close to each other in LB8 in silico model. The resulting macrocyclic peptide MP11 (SEQ ID NO: 65) was found to be slightly more potent than LB8, with a K_{d} of 465 μM (see Table 4). For comparison, the impact of a lactam bridge between K₆ side-chain NH₂ and the C-terminal carboxylate was also evaluated. The resulting peptide MP12 (SEQ ID NO: 66) yielded no appreciable binding using either NMR or MST (see Table 4).

2.2—Destabilizing and Disrupting LDH Tetramerization with Designed Macrocyclic Peptides

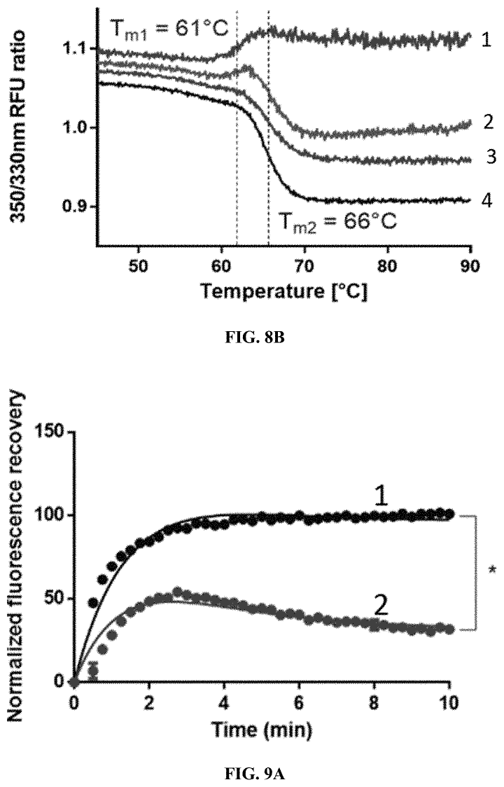

It was further tested whether macrocyclic peptides MP1 and MP7 were able to compete with N-terminal domain of native LDHB. To this end, their ability to interact with tetrameric LDH1 and LDH5 using MST was first investigated. Interestingly, macrocyclic peptide MP7, the most potent analogue, displayed an interaction at high concentrations with LDH1 and LDH5 (FIG. 8A) with a K_{d} estimated respectively at 380 μM (CI₉₅%: [315 μM to 457 μM]) and 117 μM (CI₉₅%: [94 μM to 144 μM]. Comparatively, macrocyclic peptide MP1 did not demonstrated any binding in similar conditions. This interaction between MP7 and the tetrameric protein thus suggested a displacement of LDH N-terminal arm by the cyclic peptide to reach for the tetramerization site.

The ability of macrocyclic peptides MP1 and MP7 to destabilize tetrameric LDH1 and LDH5 was further investigated. Indeed, molecules interacting at oligomeric interfaces can reduce the melting temperature of the studied oligomers owing to a perturbation of the overall stability of the complex. The impact of macrocyclic peptides MP1 and MP7 on LDH1 and LDH5 thermal denaturation was therefore evaluated using nanoDSF. While MP1 had no effect up to 500 μM on both human LDH1 and LDH5 stabilities, macrocyclic peptide MP7 induced a destabilizing conformational change on both isoforms at 400 μM (FIG. 8B). As LDH-5 is less stable than LDH1, the destabilization was stronger on the LDH-5 tetramer (ΔTm=−5° C.) than on LDH1 (ΔTm=−1.5° C.). This difference in stability of the two isozymes can besides explain the higher affinity of MP7 to LDH5 as observed by MST. The intensity of the effect was moreover dependent on protein concentration, which is coherent with the hypothesis that an increasing amount of monomers would result in a shift of the equilibrium towards the formation of tetrameric complexes. Of note, macrocyclic peptide MP7 did not induce such destabilization against a dimeric model of LDH.

Next, it was evaluated whether these macrocyclic peptides could also bind to the tetramerization site during LDH tetramer formation. Such an approach was already reported, for instance, in the case of peptides interacting at the interface of human glutathione reductase.

An experiment was thus designed to follow the recovery of LDH tetramers after a dissociation step initiated by acidic conditions. These experiments were conducted on LDH5, as it is less stable and thus more prone to dissociation than LDH1. Because strong acidic conditions (pH 2.3) are necessary to disrupt the human LDH5 (hLDH5) homotetramer, which results in partial protein denaturation, the assay was performed on rabbit LDH5 (rLDH5) that dissociates at less acidic conditions (pH 5), does not denaturate and affords reproducible data. rLDH5 shares 94% sequence identity and 98% homology with the hLDH5, with similar nanoDSF denaturation patterns. Monitoring of the rLDH5 tetrameric state was performed by following its intrinsic tryptophan fluorescence: 6 tryptophan residues can be found in each rLDHA monomer, three of them being located at the dimer-dimer interface. As the tryptophan quantum yield decreases in a polar environment, dimeric LDHs show very weak tryptophan fluorescence compared to the tetrameric form. Accordingly, dimeric rLDHA showed very weak tryptophan fluorescence at pH 5 when compared to the high fluorescence of tetrameric rLDH5 at pH 7.6. Such decay can be compared to the difference in tryptophan fluorescence between tetrameric LDH1 and dimeric LDHBtr.

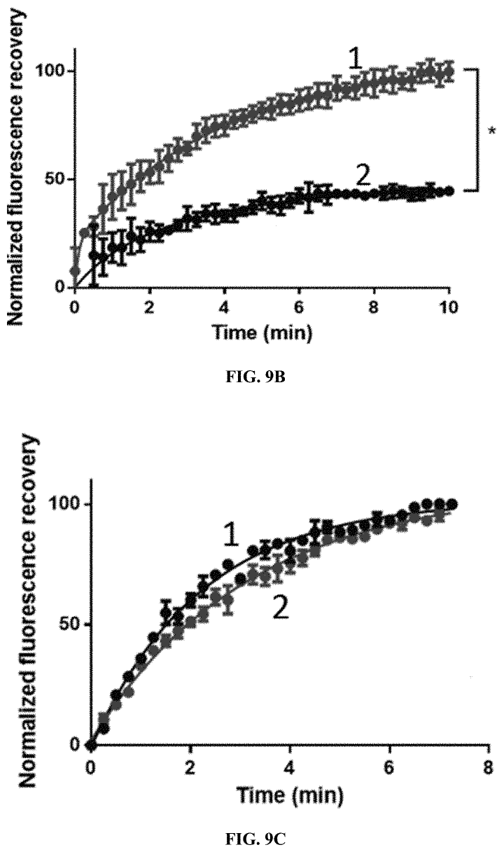

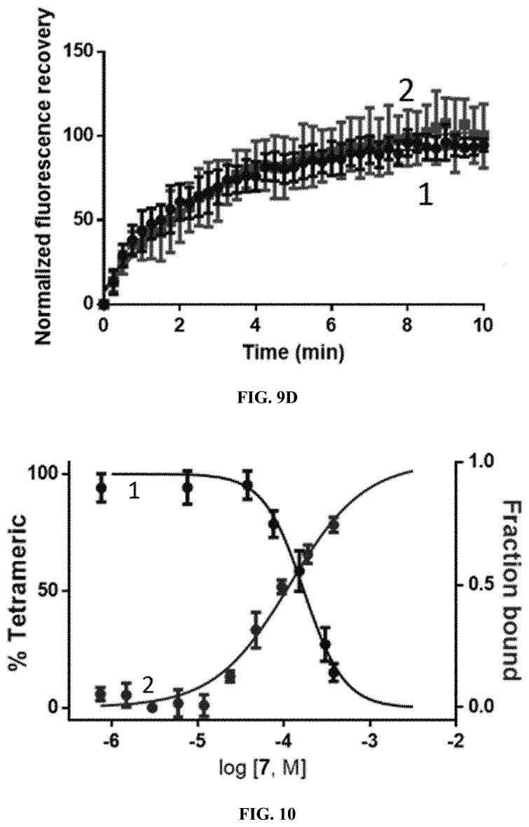

When restoring a neutral pH following acidification, macrocyclic peptides MP1 and MP7 significantly interfered with LDH5 fluorescence recovery (FIGS. 9A and 9B, respectively), while LB8 had no effect (FIG. 9C). The negative control, LBc, displayed no effect upon LDH re-association (FIG. 9D).

Finally, the ability of macrocyclic peptide MP7 to disrupt LDH oligomerization state without prior dissociation was investigated. The impact of macrocyclic peptide MP7 on the protein native fluorescence was therefore directly evaluated. Strikingly, exposing LDH1 to macrocyclic peptide MP 7 resulted in a concentration dependent conversion of LDH1 fluorescence spectrum to the one of the dimeric model LDHBtr. Normalization of the fluorescence intensity allowed to approximate the disruption ratio of LDH1 upon exposure to increasing amount of macrocyclic peptide MP7 (FIG. 10). This disruptive effect consistently matched with the interaction previously observed using MST (EC₅₀=172 μM, CI_{95%}: [142 μM to 207 μM])), suggesting that macrocyclic peptide MP7 binding to LDH tetramerization site is followed by a disruption of the protein oligomeric state. Of note, macrocyclic peptide MP7 did not induce a comparative decay of LDHBtr fluorescence spectrum.

Together, these results demonstrate that the designed cyclic peptides can target the tetramerization sites of LDHs by competing with the N-terminal domain of LDHB and LDHA monomers, leading to a destabilization and disruption of the tetrameric complexes. Moreover, these macrocyclic peptides can also interfere with the formation of LDH tetramers. These data also confirm that targeting of LDH highly conserved tetramerization site can lead to molecules interacting on both isoforms of the protein.

### Example 3

### 1—Materials and Methods

Macrocyclic peptide MP7 (SEQ ID NO: 61) was evaluated at 200 μM against Mia Paca-2 human pancreatic cancer cells (ATCC®).

The oxygen consumption rate (OCR) and extracellular acidification rate (ECAR) were measured on a Seahorse XF96 analyzer (Agilent®) with a combination of XF cell mito stress kit (Agilent®) and 2-deoxy-D-glucose (2DG; Sigma Aldrich®). Seahorse experiments were performed using 10,000 cells/well in DMEM medium with 10 mmol/L of D-glucose and 1 mmol/L of L-Glutamine Cells were incubated for 1 h in a CO₂-free incubator before analysis. In the Seahorse analyzer, oximetry was repeatedly performed in closed wells after the sequential addition of the components of the XF cell mito stress kit, namely, oligomycin to inhibit ATP-synthase, ionophore carbonyl cyanide-4-(trifluoromethoxy)phenylhydrazone (FCCP) to disrupt the mitochondrial potential, and rotenone together with antimycin A to simultaneously inhibit Complexes I and III of the mitochondrial electron transport chain (ETC). Oximetry before the addition of any agent provided the basal respiration rate of the cells; ATP-linked reparation was determined after the addition of 1 μM of oligomycin and the maximal respiration rate of the cells after the addition of 1 μM of FCCP. All data were normalized to cell numbers measured right before oximetry using a SpectraMax miniMax 300 imaging cytometer (Molecular Devices®). Macrocyclic peptide MP7 in PBS or PBS alone (control experiment) were added directly into the medium and incubated with Mia Paca-2 cells during 4 h before conducting Seahorse experiments on the medium composed of the Mia Paca-2 cells with or without macrocyclic peptide MP7.

### 2—Results

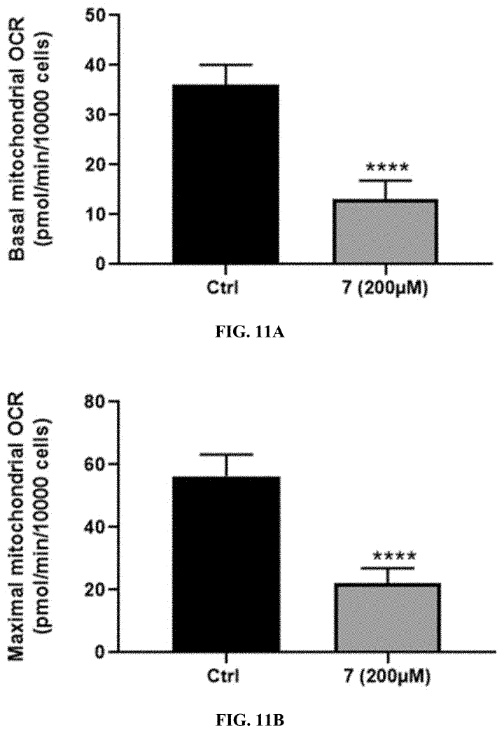

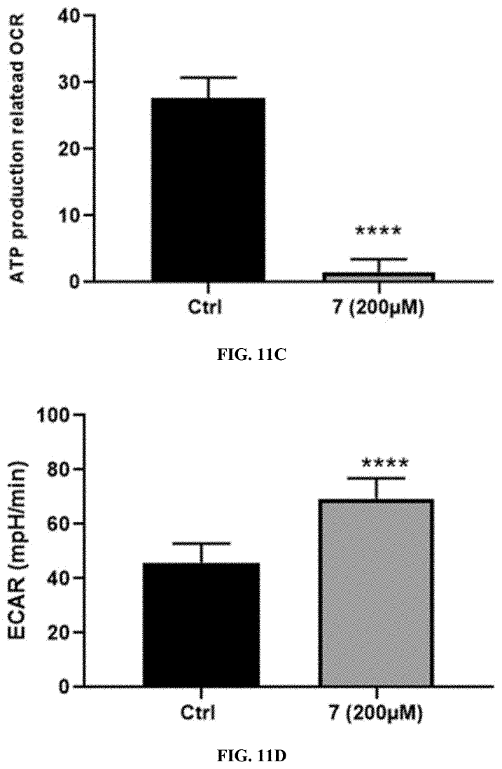

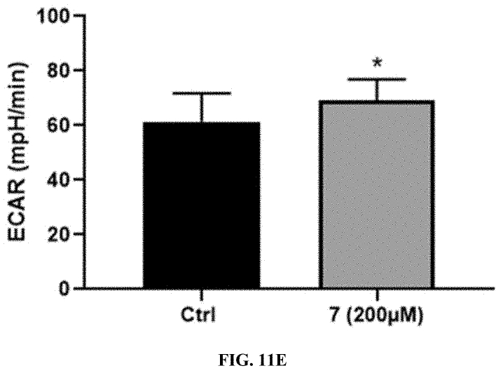

Seahorse evaluation revealed a strong decrease in mitochondrial oxygen consumption rate (OCR) as well as an increase in the glycolytic flux of Mia Paca-2 human pancreatic cancer cells (FIG. 11). In FIG. 11A, basal mitochondrial OCR represents the natural oxygen consumption rate of mitochondria in Mia Paca-2 cells in the presence of macrocyclic peptide MP7 or not (Ctrl). In FIG. 11B, maximal mitochondrial OCR represents the maximal possible oxygen consumption rate (maximal capacity) of mitochondria in Mia Paca-2 cells in the presence of macrocyclic peptide MP7 or not (Ctrl). In FIG. 11C, ATP production related to OCR represents the oxygen consumption rate directly linked to mitochondrial ATP production in Mia Paca-2 cells in the presence of macrocyclic peptide MP7 or not (Ctrl). Together, FIGS. 11A-C show that macrocyclic peptide MP7 largely inhibits Mia Paca-2 cell respiration, in such a way that mitochondria become unable to produce ATP. Because ATP provides chemical energy necessary to cancer cell life, FIG. 11A-C indicate that macrocyclic peptide MP7 has anticancer effects in Mia Paca-2 human pancreatic cancer cells. Mechanistically, it can be explained by the fact that LDH-1 catalyzes the conversion of lactate+NAD⁺ to pyruvate+NADH+H⁺, of which both pyruvate and NADH are mitochondrial fuels. If macrocyclic peptide MP7 inhibits LDH-1, then mitochondrial respiration and mitochondrial ATP production should decrease, which is exactly what is observed in FIGS. 11A-C. In FIG. 11D, ECAR represents the extracellular acidification rate linked to glycolysis when it is coupled to lactic acid fermentation, i.e., the convertion of glucose to pyruvate and then to lactate, which ends with the cell export of lactate together with protons in a 1:1 molecular ratio. ECAR is thus directly proportional to the glycolytic rate of the cells. FIG. 11E shows the maximal glycolytic capacity of the cells. When cancer cells have difficulties to produce ATP using respiration in mitochondria, they try to compensate by generating ATP using glycolysis coupled to lactic acid fermentation in the cytosol. FIGS. 11A-C showed that macrocyclic peptide MP7 inhibits the use of oxygen to produce ATP by Mia Paca-2 cancer cells. FIGS. 11D-E show that, in that case, Mia Paca-2 cells try to rescue themselves by compensating to some extent altered respiration by an increased the rate of glycolysis, hence by increasing the production of ATP by glycolysis. Altogether, FIG. 11 shows that macrocyclic peptide MP7 profoundly alters the energy metabolism of Mia Paca-2 human pancreatic cancer cells, which could induce a metabolic crisis participating in the anticancer effects of MP7.

TABLE 5
SEQUENCES
Sequences used herein

| SEQ | | |
|---|---|---|
| ID | | |
| NO: | Name | Sequences |
| 1 | hLDHA subunit | MATLKDQLIYNLLKEEQTPQNKITVVGVGAVGMACAISILMKDLA |
| | | DELALVDVIEDKLKGEMMDLQHGSLFLRTPKIVSGKDYNVTANSK |
| | | LVIITAGARQQEGESRLNLVQRNVNIFKFIIPNVVKYSPNCKLLIVSN |
| | | PVDILTYVAWKISGFPKNRVIGSGCNLDSARFRYLMGERLGVHPLS |
| | | CHGWVLGEHGDSSVPVWSGMNVAGVSLKTLHPDLGTDKDKEQW |
| | | KEVHKQVVESAYEVIKLKGYTSWAIGLSVADLAESIMKNLRRVHP |
| | | VSTMIKGLYGIKDDVFLSVPCILGQNGISDLVKVTLTSEEEARLKKS |
| | | ADTLWGIQKELQF |
| 2 | hLDHB subunit | MATLKEKLIAPVAEEEATVPNNKITVVGVGQVGMACAISILGKSLA |
| | | DELALVDVLEDKLKGEMMDLQHGSLFLQTPKIVADKDYSVTANS |
| | | KIVVVTAGVRQQEGESRLNLVQRNVNVFKFIIPQIVKYSPDCIIIVVS |
| | | NPVDILTYVTWKLSGLPKHRVIGSGCNLDSARFRYLMAEKLGIHPS |
| | | SCHGWILGEHGDSSVAVWSGVNVAGVSLQELNPEMGTDNDSEN |
| | | WKEVHKMVVESAYEVIKLKGYTNWAIGLSVADLIESMLKNLSRIH |
| | | PVSTMVKGMYGIENEVFLSLPCILNARGLTSVINQKLKDDEVAQLK |
| | | KSADTLWDIQKDLKDL |
| 3 | hLDHAtr | NKITVVGVGAVGMACAISILMKDLADELALVDVIEDKLKGEMMD |
| | | LQHGSLFLRTPKIVSGKDYNVTANSKLVIITAGARQQEGESRLNLV |
| | | QRNVNIFKFIIPNVVKYSPNCKLLIVSNPVDILTYVAWKISGFPKNR |
| | | VIGSGCNLDSARFRYLMGERLGVHPLSCHGWVLGEHGDSSVPVW |
| | | SGMNVAGVSLKTLHPDLGTDKDKEQWKEVHKQVVESAYEVIKLK |
| | | GYTSWAIGLSVADLAESIMKNLRRVHPVSTMIKGLYGIKDDVFLSV |
| | | PCILGQNGISDLVKVTLTSEEEARLKKSADTLWGIQKELQF |
| 4 | hLDHBtr | NNKITVVGVGQVGMACAISILGKSLADELALVDVLEDKLKGEMM |
| | | DLQHGSLFLQTPKIVADKDYSVTANSKIVVVTAGVRQQEGESRLN |
| | | LVQRNVNVFKFIIPQIVKYSPDCIIIVVSNPVDILTYVTWKLSGLPKH |
| | | RVIGSGCNLDSARFRYLMAEKLGIHPSSCHGWILGEHGDSSVAVW |
| | | SGVNVAGVSLQELNPEMGTDNDSENWKEVHKMVVESAYEVIKLK |
| | | GYTNWAIGLSVADLIESMLKNLSRIHPVSTMVKGMYGIENEVFLSL |
| | | PCILNARGLTSVINQKLKDDEVAQLKKSADTLWDIQKDLKDL |
| 5 | LBX | X1X2X3X4X5X6X7X8 |
| 6 | LB19 | ATLKEKLIAPVAEEEATVP |
| 7 | LB13 | ATLKEKLIAPVAE |
| 8 | LB8 | ATLKEKLI |
| 9 | LB8-A2 | ATAKEKLI |
| 10 | LB8-A3 | ATLAEKLI |
| 11 | LB8-A4 | ATLKAKLI |
| 12 | LB8-A5 | ATLKEALI |
| 13 | LB8-A7 | ATLKEKLA |
| 14 | Ac-LB8 | Ac-ATLKEKLI |
| 15 | LB8-A3-A5 | ATLAEALI |
| 16 | LB8-G1 | GTLKEKLI |
| 17 | LB8-G8 | ATLKEKLG |
| 18 | LB8-Aib1 | ATL(Aib)EKLI |
| 19 | LB8-Aib2 | ATLKE(Aib)LI |
| 20 | LB8-Aib3 | ATL(Aib)E(Aib)LI |
| 21 | LA19 | ATLKDQLIYNLLKEEQTPQ |
| 22 | LA8 | ATLKDQLI |
| 23 | LB8-A1 | AALKEKLI |
| 24 | LB8-A6 | ATLKEKAI |
| 25 | LB8-AL7 | ATLKEKL |
| 26 | LBc | Ac-LIAPVAE-NH2 |
| 27 | LB8-AcTI7 | Ac-TLKEKLI |
| 28 | LB8-TI7 | TLKEKLI |
| 29 | LB8-G3 | ATGKEKLI |
| 30 | VS-142-BisAlk | ACLKECLI |
| 31 | LT018 | CTLKCKLI |
| 32 | LT020 | ATLKCKLIC |
| 33 | LT021 | ACTLKCKLI |
| 34 | LT022 | CATLCEKLI |
| 35 | CB-09 | ATCKEKCI |
| 36 | Antennapedia | RQIKWFQNRRMKWKK |
| | Penetratin CCP | |
| 37 | TAT CCP | YGRKKRRQRRR |
| 38 | SynB1 CCP | RGGRLSYSRRRFSTSTGR |
| 39 | SynB3 CCP | RRLSYSRRRF |
| 40 | PTD-4 CCP | PIRRRKKLRRLK |
| 41 | PTD-5 CCP | RRQRRTSKLMKR |
| 42 | FHV Coat-(35- | RRRRNRTRRNRRRVR |
| | 49) CCP | |
| 43 | BMV Gag-(7- | KMTRAQRRAAARRNRWTAR |
| | 25) CCP | |
| 44 | HTLV-II Rex- | TRRQRTRRARRNR |
| | (4-16) CCP | |
| 45 | D-Tat CCP | GRKKRRQRRRPPQ |
| 46 | R9-Tat CCP | GRRRRRRRRRPPQ |
| 47 | Transportan | GWTLNSAGYLLGKINLKALAALAKKIL |
| | CCP | |
| 48 | MAP CCP | KLALKLALKLALALKLA |
| 49 | SBP CCP | MGLGLHLLVLAAALQGAWSQPKKKRKV |
| 50 | FBP CCP | GALFLGWLGAAGSTMGAWSQPKKKRKV |
| 51 | MPG CCP | GALFLGFLGAAGSTMGAWSQPKKKRKV |
| 52 | MPG(ΔNLS) | GALFLGFLGAAGSTMGAWSQPKSKRKV |
| | CCP | |
| 53 | PEP-1 CCP | KETWWETWWTEWSQPKKKRKV |
| 54 | PEP-2 CCP | KETWEETWFTEWSQPKKKRKV |
| 55 | MP1 | CTLKCKLI |
| 56 | MP2 | ACTLKCKLI |
| 57 | MP3 | ACLKECLI |
| 58 | MP4 | ATLKCKLIC |
| 59 | MPS | ATLKECLIAC |
| 60 | MP6 | CATLCEKLI |
| 61 | MP7 | CTLKCKLI |
| 62 | MP8 | CTLKCKLI |
| 63 | MP9 | CTLKCKLI |
| 64 | MP10 | Ac-KTLKDKLI |
| 65 | MP11 | ATLKEKLI |
| 66 | MP12 | ATLKEKLI |
| 67 | CT-44 | CT(mlL)KCKLI |
| 68 | CT-45 | CTLKCK(cpA)I |

### REFERENCES

Aihara et al. (2015). Synthesis of lactam-bridged cyclic peptides using sequential olefin metathesis and diimide reduction reactions. Tetrahedron 71(24): 4183-4191.
Beckert et al. (2006). Lactate stimulates endothelial cell migration. Wound Repair Regen 14(3):321-324.
Billiard et al. (2013). Quinoline 3-sulfonamides inhibit lactate dehydrogenase A and reverse aerobic glycolysis in cancer cells. Cancer Metab 1(1):19.
Brisson et al. (2016). Lactate Dehydrogenase B Controls Lysosome Activity and Autophagy in Cancer. Cancer Cell 30(3):418-431.
Colegio et al. (2014). Functional polarization of tumour-associated macrophages by tumour-derived lactic acid. Nature 513(7519):559-563.
Colen et al. (2011). Metabolic Targeting of Lactate Efflux by Malignant Glioma Inhibits Invasiveness and Induces Necrosis: An In Vivo Study. Neoplasia 13(7):620-632.
Derossi et al. (1994). The third helix of the Antennapedia homeodomain translocates through biological membranes. Biol. Chem., 269, 10444-10450.
de Saedeleer et al. (2012). Lactate Activates HIF-1 in Oxidative but Not in Warburg-Phenotype Human Tumor Cells. PLoS One 7(10).
Döbeli et al. (1982). Isoenzyme specific inhibition of the reactivation of in vitro dissociated lactic dehydrogenase isoenzymes by two different peptides isolated from human liver”, Peptides, 3(2):167-174.
Döbeli et al. (1987). Inhibition of the reactivation of acid-dissociated lactate dehydrogenase isoenzymes by their aminoterminal CNBr fragments. Peptides 8(5):773-778.
Doherty and Cleveland (2013). Targeting lactate metabolism for cancer therapeutics. J Clin Invest 123(9):3685-3692.
Eszes et al. (1996). Removal of substrate inhibition in a lactate dehydrogenase from human muscle by a single residue change. FEBS Lett 399(3):193-197.
Fischer et al. (2001). Cellular delivery of impermeable effector molecules in the form of conjugates with peptides capable of mediating membrane translocation. Bioconjug. Chem., 12, 825-841.
Fiume et al. (2014). Inhibition of lactate dehydrogenase activity as an approach to cancer therapy. Futur Med Chem 6(4):429-445.
Hewitt et al. (1999). A general method for relieving substrate inhibition in lactate dehydrogenases. Protein Eng 12(6):491-496.
Hill et al. (2014). Constraining cyclic peptides to mimic protein structure motifs. Angew Chemie—Int Ed 53(48):13020-13041.
Izumi et al. (2011). Monocarboxylate transporters 1 and 4 are involved in the invasion activity of human lung cancer cells. Cancer Sci 102(5):1007-1013.
Jafary et al. (2019). Novel Peptide Inhibitors for Lactate Dehydrogenase A (LDHA): A Survey to Inhibit LDHA Activity via Disruption of Protein-Protein Interaction”, Scientific Reports, 9(1):4686.
Jo et al. (2012). Development of alpha-helical calpain probes by mimicking a natural protein-protein interaction. J Am Chem Soc 134(42):17704-17713.
Kohlmann et al. (2013). Fragment growing and linking lead to novel nanomolar lactate dehydrogenase inhibitors. J Med Chem 56(3):1023-1040.
Labadie et al. (2015). Optimization of 5-(2,6-dichlorophenyl)-3-hydroxy-2-mercaptocyclohex-2-enones as potent inhibitors of human lactate dehydrogenase. Bioorganic Med Chem Lett 25(1):75-82.
Lacroix et al. (1998). Elucidating the folding problem of a-helices: Local motifs, long-range electrostatics, ionic strength dependence and prediction of NMR parameters. J. Mol. Biol. 284, 173-191.
Muñoz and Serrano. (1994a). Elucidating the folding problem of helical peptides using empirical parameters. Nature: Struct. Biol. 1, 399-409.
Muñoz and Serrano. (1994b). Elucidating the folding problem of a-helical peptides using empirical parameters, II. Helix macrodipole effects and rational modification of the helical content of natural peptides. J. Mol. Biol 245, 275-296.
Muñoz and Serrano. (1994c). Elucidating the folding problem of a-helical peptides using empirical parameters III: Temperature and pH dependence. J. Mol. Biol 245, 297-308.
Muñoz and Serrano. (1997). Development of the Multiple Sequence Approximation within the Agadir Model of a-Helix Formation. Comparison with Zimm-Bragg and Lifson-Roig Formalisms. Biopolymers 41, 495-509.
Purkey et al. (2016) Cell Active Hydroxylactam Inhibitors of Human Lactate Dehydrogenase with Oral Bioavailability in Mice. ACS Med Chem Lett 7(10):896-901.
Rai et al. (2017). Discovery and Optimization of Potent, Cell-Active Pyrazole-Based Inhibitors of Lactate Dehydrogenase (LDH). J Med Chem 60(22):9184-9204.
Rani and Kumar (2016). Recent Update on Human Lactate Dehydrogenase Enzyme 5 (hLDH5) Inhibitors: A Promising Approach for Cancer Chemotherapy. J Med Chem 59(2):487-496.
Rao and Rossmann (1973). Comparison of super-secondary structures in proteins. J Mol Biol 76(2):241-250.
Remington's Pharmaceutical Sciences 17th Ed., Mack Publishing Co. (A. R. Gennaro ed. 1985).
Rudolph and Jaenicke (1976). Kinetics of reassociation and reactivation of pig-muscle lactic dehydrogenase after acid dissociation. Eur J Biochem 63(2):409-17.
Sonveaux et al. (2008). Targeting lactate-fueled respiration selectively kills hypoxic tumor cells in mice. J Clin Invest 118(12):3930-3942.
Spokoyny et al. (2013). Pentelute, A perfluoroaryl-cysteine SNAr chemistry approach to unprotected peptide stapling, J. Am. Chem. Soc. 135:5946-5949.
Taylor (2002). The synthesis and study of side-chain lactam-bridged peptides. Biopolymers. 66(1):49-75.
Végran et al. (2011). Lactate influx through the endothelial cell monocarboxylate transporter MCT1 supports an NF-kB/IL-8 pathway that drives tumor angiogenesis. Cancer Res 71(7):2550-2560.
Vives et al. (1997). A truncated HIV-1 Tat protein basic domain rapidly translocates through the plasma membrane and accumulates in the cell nucleus. J. Biol. Chem., 272, 16010-16017.
Ward et al. (2012). Design and Synthesis of Novel Lactate Dehydrogenase A Inhibitors by Fragment-Based Lead Generation. J Med Chem 55:3285-3306.
Yachdav et al. (2014). PredictProtein—an open resource for online prediction of protein structural and functional features. Nucleic acids research, gku366.
Ždralević et al. (2018). Double genetic disruption of lactate dehydrogenase A and B is required to ablate the “Warburg effect” restricting tumor growth to oxidative metabolism. J Biol Chem (49).

1-15. (canceled)

16. A polypeptide that inhibits the tetramerization of the lactate dehydrogenase subunits, said polypeptide comprising the amino acid sequence of formula (I)
X1-X2-X3-X4-X5-X6-X7-X8 (I) (SEQ ID NO: 5),
wherein:
X1 represents any amino acid residue, preferentially selected from the group consisting of amino acid residues A, G, K and C;
X2 represents C, T or S;
X3 represents C, L, A, T, cpA (cyclopropyl-L-alanine), chG (L-cyclohexylglycine), chA (cyclohexyl-L-alanine) or mlL (γ-Methyl-L-leucine);
X4 represents any amino acid residue, preferentially a positively charged or neutral amino acid residue, preferentially selected from the group consisting of amino acid residues K, C, A and Aib (2-aminoisobutyric acid), and more preferentially amino acid K;
X5 represents any amino acid residue, preferentially a negatively or positively charged or neutral amino acid residue, preferentially selected from the group consisting of amino acid residues E, D, K, A and C, and more preferentially amino acid E;
X6 represents any amino acid residue, preferentially a negatively or positively charged or neutral amino acid residue, preferentially selected from the group consisting of amino acid residues E, K, Q, A, Aib (2-aminoisobutyric acid) and C, and more preferentially amino acid K;
X7 represents C, L, I, cpA (cyclopropyl-L-alanine), chG (L-cyclohexylglycine), chA (cyclohexyl-L-alanine) or mlL (γ-methyl-L-leucine); and
X8 represents C, I or G.

17. The polypeptide according to claim 16, wherein said polypeptide is a linear polypeptide.

18. The polypeptide according to claim 16, wherein said polypeptide is a linear polypeptide comprising an amino acid sequence selected from the group consisting of SEQ ID NO: 6 to SEQ ID NO: 22.

19. The polypeptide according to claim 16, wherein said polypeptide is a cyclic polypeptide.

20. The polypeptide according to claim 16, wherein said polypeptide is a cyclic polypeptide comprising an amino acid sequence selected from the group consisting of SEQ ID NO: 30 to SEQ ID NO: 35, SEQ ID NO: 55 to SEQ ID NO: 58, SEQ ID NO: 61 to SEQ ID NO: 65, SEQ ID NO: 67 and SEQ ID NO: 68.

21. The polypeptide according to claim 16, wherein said polypeptide is a cyclic polypeptide comprising an amino acid sequence selected from the group consisting of SEQ ID NO: 55, SEQ ID NO: 61, SEQ ID NO: 62 and SEQ ID NO: 63, SEQ ID NO: 67 and SEQ ID NO: 68.

22. The polypeptide according to claim 16, wherein said polypeptide is a cyclic polypeptide comprising an amino acid sequence represented by SEQ ID NO: 61, SEQ ID NO: 67 or SEQ ID NO: 68.

23. The polypeptide according to claim 16, wherein said lactate dehydrogenase subunit is lactate dehydrogenase B (LDHB) subunit.

24. The polypeptide according to claim 16, wherein the —OH group of the free —COOH group of the last amino acid residue at the C-terminus of the polypeptide is replaced by a group selected from an —O-alkyl group, an —O-aryl group, a —NH₂ group, a —N-alkyl amine group, a —N-aryl amine group or a —N-alkyl/aryl group.

25. A polynucleotide encoding a polypeptide according to claim 16.

26. A pharmaceutical composition comprising at least one polypeptide according to claim 16, and at least one pharmaceutically acceptable vehicle.

27. A kit for preventing and/or treating a cancer comprising at least one polypeptide according to claim 16, a polynucleotide encoding said polypeptide, or a pharmaceutical composition comprising said polypeptide with at least one pharmaceutically acceptable vehicle, and optionally an anticancer agent.

28. A medicament comprising a polypeptide according to claim 16, a polynucleotide encoding said polypeptide, or a pharmaceutical composition comprising said polypeptide with at least one pharmaceutically acceptable vehicle.

29. A method for preventing and/or treating a cancer in a subject in need thereof comprising the step of administering to the subject an effective amount of a polypeptide according to claim 16, a polynucleotide encoding said polypeptide, or a pharmaceutical composition comprising said polypeptide with at least one pharmaceutically acceptable vehicle.

30. A method for screening a compound affecting the tetramerization of the lactate dehydrogenase subunits comprising the steps of:
a. providing a system comprising truncated lactate dehydrogenase (LDHtr) subunit;
b. providing the system with a candidate compound modulating the activity of a native tetrameric LDH; and
c. measuring a level of binding of the candidate compound to a dimer of LDHtr subunits in the presence or in the absence of a polypeptide according to claim 16;
wherein the observation of a competition between the polypeptide and the candidate compound for the binding to the dimer of LDHtr subunits is indicative of the candidate compound being an inhibitor of the tetramerization of the lactate dehydrogenase subunits.

31. The method according to claim 30, wherein the observation of a competition between the polypeptide and the candidate compound for the binding to the LDHtr subunit is indicative of the specificity of the binding of the candidate compound towards the tetramerization site onto the lactate dehydrogenase subunits.

32. A method for screening a compound affecting the tetramerization of the lactate dehydrogenase subunits comprising the steps of:
a) providing a system (1) comprising truncated lactate dehydrogenase (LDHtr) subunits and a system (2) comprising native tetrameric LDH;
b) providing the systems (1) and (2) with a candidate compound modulating the activity of a native tetrameric LDH; and
c) measuring a level of binding (Kd) of the candidate compound to a dimer of LDHtr subunits in system (1) and to a native tetrameric LDH in system (2);
wherein the observation of a binding of the candidate compound to the dimer of LDHtr subunits in system (1) and wherein the observation of an altered binding of the candidate compound to the native tetrameric LDH in system (2) are indicative of the candidate compound being an inhibitor of the tetramerization of the lactate dehydrogenase subunits, by interacting at the surface of the LDH subunits.


SEQUENCE LISTING (68 sequences)

SEQ ID NO: 1 - protein, 332 aa, Homo sapiens
  hLDHA subunit
MATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 2 - protein, 334 aa, Homo sapiens
  hLDHB subunit
MATLKEKLIAPVAEEEATVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 3 - protein, 312 aa, Artificial Sequence
  hLDHAtr
NKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

SEQ ID NO: 4 - protein, 314 aa, Artificial Sequence
  hLDHBtr
NNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCIIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVESAYEVIKLKGYTNWAIGLSVADLIESMLKNLSRIHPVSTMVKGMYGIENEVFLSLPCILNARGLTSVINQKLKDDEVAQLKKSADTLWDIQKDLKDL

SEQ ID NO: 5 - protein, 8 aa, Artificial Sequence
  SITE 1: X represents any amino acid residue, preferentially selected from the group consisting of amino acid residues A, G, K and C
  LBX
  SITE 2: X represents C, T or S
  SITE 3: X represents C, L, A, T, cpA (cyclopropyl-L- alanine), chG (L-cyclohexylglycine), chA (cyclohexyl-L-alanine) or mlL (gamma-methyl-L-leucine)
  SITE 4: X represents any amino acid residue, preferentially a positively charged or neutral amino acid residue, preferentially selected from the group consisting of amino acid residues K, C, A and Aib (2-aminoisobutyric acid),and more preferentially amino acid K
  SITE 5: X represents any amino acid residue, preferentially a  negatively or positively charged or neutral amino acid residue, preferentially selected from the group consisting of amino acid residues E, D, K, A and C, and more preferentially amino acid E
  SITE 6: X represents any amino acid residue, preferentially a negatively or positively charged or neutral amino acid residue, preferentially selected fr om the group consisting of amino acid residues E, K, Q, A, Aib (2-am inoisobutyric acid) and C, and more preferent
  SITE 7: X represents C, L, I, cpA (cyclopropyl-L- alanine), chG (L-cyclohexylglycine), chA (cyclohexyl-L-alanine) or mlL (gamma-methyl-L-leucine)
  SITE 8: X represents C, I or G
XXXXXXXX

SEQ ID NO: 6 - protein, 19 aa, Artificial Sequence
  LB19
ATLKEKLIAPVAEEEATVP

SEQ ID NO: 7 - protein, 13 aa, Artificial Sequence
  LB13
ATLKEKLIAPVAE

SEQ ID NO: 8 - protein, 8 aa, Artificial Sequence
  LB8
ATLKEKLI

SEQ ID NO: 9 - protein, 8 aa, Artificial Sequence
  LB8-A2
ATAKEKLI

SEQ ID NO: 10 - protein, 8 aa, Artificial Sequence
  LB8-A3
ATLAEKLI

SEQ ID NO: 11 - protein, 8 aa, Artificial Sequence
  LB8-A4
ATLKAKLI

SEQ ID NO: 12 - protein, 8 aa, Artificial Sequence
  LB8-A5
ATLKEALI

SEQ ID NO: 13 - protein, 8 aa, Artificial Sequence
  LB8-A7
ATLKEKLA

SEQ ID NO: 14 - protein, 8 aa, Artificial Sequence
  MOD_RES 1: ACETYLATION
  Ac-LB8
ATLKEKLI

SEQ ID NO: 15 - protein, 8 aa, Artificial Sequence
  LB8-A3-A5
ATLAEALI

SEQ ID NO: 16 - protein, 8 aa, Artificial Sequence
  LB8-G1
GTLKEKLI

SEQ ID NO: 17 - protein, 8 aa, Artificial Sequence
  LB8-G8
ATLKEKLG

SEQ ID NO: 18 - protein, 8 aa, Artificial Sequence
  LB8-Aib1
  MOD_RES 4: aib
  MOD_RES 4: Aib
ATLXEKLI

SEQ ID NO: 19 - protein, 8 aa, Artificial Sequence
  LB8-Aib2
  MOD_RES 6: Aib
ATLKEXLI

SEQ ID NO: 20 - protein, 8 aa, Artificial Sequence
  LB8-Aib3
  MOD_RES 4: Aib
  MOD_RES 6: Aib
ATLXEXLI

SEQ ID NO: 21 - protein, 19 aa, Artificial Sequence
  LA19
ATLKDQLIYNLLKEEQTPQ

SEQ ID NO: 22 - protein, 8 aa, Artificial Sequence
  LA8
ATLKDQLI

SEQ ID NO: 23 - protein, 8 aa, Artificial Sequence
  LB8-A1
AALKEKLI

SEQ ID NO: 24 - protein, 8 aa, Artificial Sequence
  LB8-A6
ATLKEKAI

SEQ ID NO: 25 - protein, 7 aa, Artificial Sequence
  LB8-AL7
ATLKEKL

SEQ ID NO: 26 - protein, 7 aa, Artificial Sequence
  MOD_RES 1: ACETYLATION
  LBc
  MOD_RES 7: AMIDATION
LIAPVAE

SEQ ID NO: 27 - protein, 7 aa, Artificial Sequence
  MOD_RES 1: ACETYLATION
  LB8-AcTI7
TLKEKLI

SEQ ID NO: 28 - protein, 7 aa, Artificial Sequence
  LB8-TI7
TLKEKLI

SEQ ID NO: 29 - protein, 8 aa, Artificial Sequence
  LB8-G3
ATGKEKLI

SEQ ID NO: 30 - protein, 8 aa, Artificial Sequence
  VS-142-BisAlk (linker alpha, aplpha prime bisbromoxylene)
ACLKECLI

SEQ ID NO: 31 - protein, 8 aa, Artificial Sequence
  LT018 (linker alpha, aplpha prime bisbromoxylene)
CTLKCKLI

SEQ ID NO: 32 - protein, 9 aa, Artificial Sequence
  LT020 (linker alpha, aplpha prime bisbromoxylene)
ATLKCKLIC

SEQ ID NO: 33 - protein, 9 aa, Artificial Sequence
  LT021 (linker alpha, aplpha prime bisbromoxylene)
ACTLKCKLI

SEQ ID NO: 34 - protein, 9 aa, Artificial Sequence
  LT022 (linker alpha, aplpha prime bisbromoxylene)
CATLCEKLI

SEQ ID NO: 35 - protein, 8 aa, Artificial Sequence
  CB-09 (linker alpha, aplpha prime bisbromoxylene)
ATCKEKCI

SEQ ID NO: 36 - protein, 15 aa, Artificial Sequence
  Antennapedia Penetratin CCP
RQIKWFQNRRMKWKK

SEQ ID NO: 37 - protein, 11 aa, Artificial Sequence
  TAT CCP
YGRKKRRQRRR

SEQ ID NO: 38 - protein, 18 aa, Artificial Sequence
  SynB1 CCP
RGGRLSYSRRRFSTSTGR

SEQ ID NO: 39 - protein, 10 aa, Artificial Sequence
  SynB3 CCP
RRLSYSRRRF

SEQ ID NO: 40 - protein, 12 aa, Artificial Sequence
  PTD-4 CCP
PIRRRKKLRRLK

SEQ ID NO: 41 - protein, 12 aa, Artificial Sequence
  PTD-5 CCP
RRQRRTSKLMKR

SEQ ID NO: 42 - protein, 15 aa, Artificial Sequence
  FHV Coat-(35-49) CCP
RRRRNRTRRNRRRVR

SEQ ID NO: 43 - protein, 19 aa, Artificial Sequence
  BMV Gag-(7-25) CCP
KMTRAQRRAAARRNRWTAR

SEQ ID NO: 44 - protein, 13 aa, Artificial Sequence
  HTLV-II Rex-(4-16) CCP
TRRQRTRRARRNR

SEQ ID NO: 45 - protein, 13 aa, Artificial Sequence
  D-Tat CCP
GRKKRRQRRRPPQ

SEQ ID NO: 46 - protein, 13 aa, Artificial Sequence
  R9-Tat CCP
GRRRRRRRRRPPQ

SEQ ID NO: 47 - protein, 27 aa, Artificial Sequence
  Transportan CCP
GWTLNSAGYLLGKINLKALAALAKKIL

SEQ ID NO: 48 - protein, 17 aa, Artificial Sequence
  MAP CCP
KLALKLALKLALALKLA

SEQ ID NO: 49 - protein, 27 aa, Artificial Sequence
  SBP CCP
MGLGLHLLVLAAALQGAWSQPKKKRKV

SEQ ID NO: 50 - protein, 27 aa, Artificial Sequence
  FBP CCP
GALFLGWLGAAGSTMGAWSQPKKKRKV

SEQ ID NO: 51 - protein, 27 aa, Artificial Sequence
  MPG CCP
GALFLGFLGAAGSTMGAWSQPKKKRKV

SEQ ID NO: 52 - protein, 27 aa, Artificial Sequence
  MPG(deltaNLS) CCP
GALFLGFLGAAGSTMGAWSQPKSKRKV

SEQ ID NO: 53 - protein, 21 aa, Artificial Sequence
  PEP-1 CCP
KETWWETWWTEWSQPKKKRKV

SEQ ID NO: 54 - protein, 21 aa, Artificial Sequence
  PEP-2 CCP
KETWFETWFTEWSQPKKKRKV

SEQ ID NO: 55 - protein, 8 aa, Artificial Sequence
  MP1 (linker m-benzyl)
CTLKCKLI

SEQ ID NO: 56 - protein, 9 aa, Artificial Sequence
  MP2 (linker m-benzyl)
ACTLKCKLI

SEQ ID NO: 57 - protein, 8 aa, Artificial Sequence
  MP3 (linker m-benzyl)
ACLKECLI

SEQ ID NO: 58 - protein, 9 aa, Artificial Sequence
  MP4 (linker m-benzyl)
ATLKCKLIC

SEQ ID NO: 59 - protein, 10 aa, Artificial Sequence
  MP5 (linker m-benzyl)
ATLKECLIAC

SEQ ID NO: 60 - protein, 9 aa, Artificial Sequence
  MP6 (linker m-benzyl)
CATLCEKLI

SEQ ID NO: 61 - protein, 8 aa, Artificial Sequence
  MP7 (linker p-tetrafluorophenyl)
CTLKCKLI

SEQ ID NO: 62 - protein, 8 aa, Artificial Sequence
  MP8 (linker o-benzyl)
CTLKCKLI

SEQ ID NO: 63 - protein, 8 aa, Artificial Sequence
  MP9 (linker p-benzyl)
CTLKCKLI

SEQ ID NO: 64 - protein, 8 aa, Artificial Sequence
  MOD_RES 1: amino acid residue 1 acetylated ACETYLATION
  MP10 (linker Lactam bridge K1-D5)
KTLKDKLI

SEQ ID NO: 65 - protein, 8 aa, Artificial Sequence
  MP11 (linker Lactam bridge Nter-E5)
ATLKEKLI

SEQ ID NO: 66 - protein, 8 aa, Artificial Sequence
  MP12 (linker Lactam bridge K6-Cter)
ATLKEKLI

SEQ ID NO: 67 - protein, 8 aa, Artificial Sequence
  CT-44 (linker p-tetrafluorophenyl)
  VARIANT 3: X represents mlL (gammma-methyl-L-leucine)
CTXKCKLI

SEQ ID NO: 68 - protein, 8 aa, Artificial Sequence
  CT-45 (linker p-tetrafluorophenyl)
  VARIANT 7: X represents cpA (cyclopropyl-L-alanine)
CTLKCKXI